SEQUENCE LISTING (3 sequences)

SEQ ID NO: 1 - protein, 31 aa, Artificial Sequence
  F3 Peptide
KDEPQRRSARLSAKPAPPKPEPKPKKAPAKK

SEQ ID NO: 2 - protein, 32 aa, Artificial Sequence
  F3-C Peptide
KDEPQRRSARLSAKPAPPKPEPKPKKAPAKKC

SEQ ID NO: 3 - protein, 32 aa, Artificial Sequence
  C-F3 Peptide
CKDEPQRRSARLSAKPAPPKPEPKPKKAPAKK

(19) United States
(12) Patent Application Publication
(10) Pub. No.: US 2005/0196343 A1
(43) Pub. Date: Sep. 8, 2005

(54) Degradable nanoparticles

(75) Inventors: G. Ramachandra Reddy, Novi, MI (US); Nandanan Erathodiyil, Singapore (SG)

Correspondence Address: STERNE, KESSLER, GOLDSTEIN & FOX PLLC, 1100 NEW YORK AVENUE, N.W., WASHINGTON, DC, 20005, US

(73) Assignee: Molecular Therapeutics, Inc., Ann Arbor, MI (US)

(21) Appl. No.: 11/066,566

(22) Filed: Feb. 28, 2005

Related U.S. Application Data
(60) Provisional application No. 60/548,105, filed on Feb. 27, 2004.

(51) Int. Cl. A61K 49/00; A61K 38/17; A61K 31/7072

(52) U.S. Cl. 424/9.322; 514/12; 424/489; 514/49

(57) ABSTRACT

The present invention relates to polymeric nanoparticles, particularly useful in drug and agent delivery, as well as for imaging and diagnosis. The polymeric nanoparticles of the present invention comprise cross-linkers that, when degraded, leave simple linear polymeric molecules that can be excreted by the body. The present invention also relates to methods of producing the polymeric nanoparticles of the present invention, and methods of using them in drug and agent delivery, as well as imaging and diagnosis.

### CROSS REFERENCE TO RELATED APPLICATIONS

The present application claims the benefit of the filing date of U.S. Provisional Patent Application No. 60/548,105, filed Feb. 27, 2004, the disclosure of which application is incorporated by reference herein in its entirety.

### BACKGROUND OF THE INVENTION

1. Field of the Invention

The present invention relates to polymeric nanoparticles, particularly useful in drug and agent delivery, as well as for imaging and diagnosis. The polymeric nanoparticles of the present invention comprise cross-linkers that, when degraded, leave simple, linear polymeric molecules that can be excreted by the body. The present invention also relates to methods of producing the polymeric nanoparticles of the present invention, and methods of using them in drug and agent delivery, as well as imaging and diagnosis.

2. Related Art

U.S. Pat. No. 6,143,558 to Kopelman et al., describes polymeric nanoparticles for use as optical probes for monitoring the response of cells to various external stimuli and insults. The nanoparticles of the '558 patent are designed to not be biodegradable and retain their contents, thereby allowing external monitoring of cellular responses.

Due to their small size, polymeric nanoparticles have been found to evade recognition and uptake by the reticulo-endothelial system (RES), and thus can circulate in the blood for an extended period. (Borchard, G. et al., Pharm Res. 7:1055-1058 (1996)). In addition, nanoparticles are able to extravasate at the pathological site, such as the leaky vasculature of a solid tumor, providing a passive targeting mechanism. (Yuan F. et al., Cancer Research 55:3752-3756 (1995); Duncan, R. et al., STP Pharma. Sci. 4:237 (1996).) U.S. Pat. No. 6,322,817 to Maitra et al., discloses the production of nanoparticles comprised of polymeric micelles containing the anticancer drug paclitaxel. The '817 patent describes the use of amphiphilic monomers in conjunction with a cross-linking agent to create the encapsulating micelles. The cross-linking agents disclosed in the '817 patent however, are not biodegradable.

U.S. Pat. No. 6,521,431 describes several biodegradable cross-linkers that can be used in the preparation of biodegradable nanoparticles.

An important feature of any nanoparticle, especially for agent delivery, is the biocompatibility of the particle. This requires that the polymer particle degrade after some period so that it can be excreted. These criteria require polymer compositions that are well tolerated. In addition, controlled polymer degradation also allows for increased levels of agent delivery to a diseased site.

However, to date there remain few degradable nanoparticles composed of well-tolerated polymers. The present invention fulfills this need by providing cross-linked polymeric nanoparticles that degrade into simple linear polymeric molecules that can be easily excreted from the body. The nanoparticles of the present invention can be used for patient diagnosis, treatment and imaging, and the degradable nature of the nanoparticles allow them to deliver enhanced amounts of encapsulated contents at the disease site.

### SUMMARY OF THE INVENTION

In one embodiment, the present invention provides polymeric nanoparticles comprising: (a) a backbone polymer selected from the group consisting of poly(acrylamide), poly(2-hydroxyethyl methacrylate), poly(glycerol monomethacrylate), poly(acrylic acid), poly((aminoalkyl)methacrylamides), poly(sodium acrylate), poly(vinyl pyrrolidone) and mixtures thereof; and (b) a polymeric cross-linker selected from the group consisting of glycerol(bis)acrylate, 3-(acryloyloxy)-2-hydroxypropyl methacrylate, ethylene glycol diacrylate, glycerol dimethacrylate, divinyl citrate and mixtures thereof, wherein the polymeric cross-linker links two or more of the backbone polymers. Suitably, the nanoparticle of the present invention is biodegradable, and is less than 200 nm in diameter. Suitable backbone polymers for use in the practice of the present invention include poly(acrylamide), poly(3-(aminopropyl)methacrylamide), poly(vinyl pyrrolidone) and poly(acrylic acid).

The polymeric nanoparticles of the present invention can further comprise a functionalized surface group, including an amine group, and can further comprise targeting molecules such as Herceptin and antibodies bound to their surface. In certain embodiments, the nanoparticles can comprise F3 peptides conjugated to their surface. The nanoparticles of the present invention can suitably encapsulate one or more water-soluble, or water-insoluble agents, including, but not limited to, a small organic molecule drug, a DNA molecule, an RNA molecule, a protein, a fluorescent dye, a radioisotope, a contrast agent, a degradable polymer and an imaging agent. In suitable embodiments, the nanoparticles of the present invention can comprise two or more agents. Suitable water-soluble agents include iron oxide, gemcitabine and photofrin.

Suitable polymeric nanoparticles include nanoparticles where the backbone polymer is poly(acrylamide) and the polymeric cross-linker is 3-(acryloyloxy)-2-hydroxypropyl methacrylate, where the backbone polymer is a mixture of poly(acrylamide) and poly(3-(aminopropyl)methacrylamide) (or suitable variants) and the polymeric cross-linker is 3-(acryloyloxy)-2-hydroxypropyl methacrylate, where the backbone polymer is a mixture of poly(acrylamide) and poly(acrylic acid) and the polymeric cross-linker is 3-(acryloyloxy)-2-hydroxypropyl methacrylate, where the backbone polymer is a mixture of poly(acrylamide) and poly(3-(aminopropyl)methacrylamide) and the polymeric cross-linker is glycerol(bis)acrylate, and where the backbone polymer is a mixture of poly(acrylamide) and poly(acrylic acid) and the polymeric cross-linker is glycerol(bis)acrylate.

The present invention also provides methods of producing polymeric nanoparticles comprising: (a) forming a solution of polymeric monomers and cross-linkers, wherein the monomers are selected from the group consisting of acrylamide, (aminoalkyl)methacrylamide, 2-hydroxyethyl methacrylate, glycerol monomethacrylate, acrylic acid, sodium acrylate, vinyl pyrrolidone and mixtures thereof, and the cross-linkers are selected from the group consisting of glycerol(bis)acrylate, 3-(acryloyloxy)-2-hydroxypropyl methacrylate, ethylene glycol diacrylate, glycerol dimethacrylate, divinyl citrate and mixtures thereof; (b) initiating polymerization to generate a solid particle; and (c) removing the solid particle from solution. These solid particles can then be passed through one or more porous filters to generate nanoparticles less than 200 nm in diameter. The methods of the present invention can further comprise encapsulating agents within the nanoparticles of the present invention and adding functional groups to their surface. In suitable embodiments, the polymerization will take place in the presence of one or more surfactants, or similar molecules.

The present invention also provides methods of controlling the rate of degradation (suitably biodegradation) by changing the cross-linking ratio of a cross-linker relative to the backbone monomer concentration. Varying the amount (e.g., 5%, 10%, 15%, 20%, 25% or 30%) of cross-linker relative to backbone monomers (i.e., the density of the cross-linker relative to the backbone monomers) can modulate the release rate of encapsulated drug.

The polymeric nanoparticles of the present invention can further comprise a functionalized surface group, including, but not limited to, carboxylic acid or amine groups, and can further comprise targeting molecules such as antibodies and cancer specific peptides on their surface. In suitable embodiments, the nanoparticles of the present invention can comprise two or more agents encapsulated within the nanoparticle.

The present invention also provides methods of attaching specific targeting agents (suitably peptides) to nanoparticles through the use of a cysteine linker. The present invention can further comprise attaching small molecules, e.g., haptens, for cancer cell targeting. In certain embodiments, the target agents are peptides selected from the group consisting of SEQ ID NO. 1, SEQ ID NO: 2 and SEQ ID NO: 3.

The polymeric nanoparticles of the present invention can also encapsulate degradable polymers, including, but not limited to, polyesters such as poly(lactic-glycolic acid) PLGA, polysorbitol, polysorbitol-adipate, polymannitol polymers, poly amino acids such as polyaspartic acid, polylysine and polyglutamic acid. These polymers can be used to further control the release or retention of a drug, imaging agent or other encapsulated agent. In suitable embodiments, the degradable polymers are co-encapsulated with a second agent.

In another embodiment, the present invention provides methods of treating a tumor in a mammalian patient comprising administering to the patient a polymeric nanoparticle according to the present invention, wherein the polymeric nanoparticle encapsulates one or more cancer chemotherapeutic agents such as gemcitabine or photofrin. In suitable embodiments, the nanoparticle can further encapsulate an imaging agent such as iron oxide so that the nanoparticle can be imaged. The present invention also provides methods of imaging the polymeric nanoparticles which encapsulate imaging agents.

The present invention also provides methods of treating a tumor in a mammalian patient comprising administering to the patient a polymeric nanoparticle according to the present invention and administering ionizing radiation to the patient, wherein the polymeric nanoparticle encapsulates one or more radiation-sensitizing agents. Suitable radiation-sensitizing agents include, but are not limited to, gemcitabine, paclitaxel and carboplatin. The nanoparticles can also comprise an imaging agent to allow for imaging of the nanoparticles in the patient.

The present invention also provides polymeric nanoparticles comprising a backbone polymer selected from the group consisting of poly(acrylamide), poly(acrylic acid), poly(3-(aminopropyl)methacrylamide) and mixtures thereof, cross-linked with; about 10% glycerol(bis)acrylate cross-linker; and a functionalized surface group conjugated to a peptide selected from the group consisting of SEQ ID NO: 1, SEQ ID NO: 2 and SEQ ID NO: 3, wherein the polymeric nanoparticle encapsulates iron oxide. In suitable embodiments, the polymeric nanoparticles can comprise about 20% glycerol(bis)acrylate cross-linker. In other embodiments, the nanoparticles can comprise about 10% or about 20% 3-(acryloyloxy)-2-hydroxypropyl methacrylate cross-linker. The nanoparticles can also further encapsulate gemcitabine.

In another embodiment, the present invention provides polymeric nanoparticles produced by the process comprising: forming a solution of polymeric monomers and cross-linkers, the monomers selected from the group consisting of acrylamide, 3-(aminopropyl)methacrylamide, acrylic acid, and mixtures thereof; the cross-linkers selected from the group consisting of glycerol(bis)acrylate, 3-(acryloyloxy)-2-hydroxypropyl methacrylate and mixtures thereof; optionally adding a functionalized monomer; adding iron oxide; initiating polymerization to generate a solid particle, the particle comprising a polymeric backbone of the polymeric monomers cross-linked with the polymeric cross-linker; conjugating a peptide selected from the group consisting of SEQ ID NO: 1, SEQ ID NO: 2 and SEQ ID NO: 3 to a functionalized surface group on the nanoparticle; and removing the nanoparticle from solution, wherein the cross-linker density is about 10% relative to the polymeric backbone. In suitable embodiments, gemcitabine can be added prior to initiation of polymerization. In other embodiments, the cross-linker density can be about 20% relative to the polymeric backbone.

In another embodiment, the present invention provides methods of producing a radio-labeled polymeric nanoparticle comprising: (a) ¹⁴C radiolabeled acrylamide and (b) ¹⁴C radiolabeled glycerol dimethacrylate.

### BRIEF DESCRIPTION OF THE FIGURES

FIGS. 1A-1B show (A) Multi Angle Light Scattering and (B) a scanning electron micrograph (SEM) of polymeric nanoparticles (amine-functionalized, 20% AHM nanoparticles encapsulating Photofrin and SPIO) in accordance with the present invention.

FIG. 2 is a representation of a suitable backbone polymer with polymer cross-linkers according to one embodiment of the present invention.

FIG. 3 is a representation of possible degradation end products produced after degradation of the configuration shown in FIG. 2.

FIG. 4A shows the percentage of Ruthenium dye remaining in glycerol (bis)acrylate nanoparticles following treatment with NaOH and filtration after a 12 hour incubation.

FIG. 4B shows the percentage of Photofrin eluted after a 12 hour incubation following treatment with NaOH and filtration from glycerol(bis) acrylate nanoparticles (squares) and nanoparticles made with a non-biodegradable cross-linker (N,N-methylene (bis)acrylamide) and acrylamide as the polymer backbone (diamonds).

FIGS. 5A and 5B show the effect of Ruthenium dye (Ru) encapsulated in glycerol(bis)acrylate nanoparticles in laser assisted cell kill of Rat 9L glioma cells. 5A shows the effects of blank glycerol(bis)acrylate nanoparticles and laser light. 5B shows the effects of Ru dye encapsulated in glycerol(bis)acrylate nanoparticles and laser light.

FIGS. 6a-6f show images over 6 hours after intravenous injection of the iron oxide loaded glycerol(bis)acrylate nanoparticles into a 9L tumor bearing rat. (a) Before injection; (b-f) after injection.

FIG. 7 shows the percent degradation over time of Ru-dye-encapsulated citrate-poly(vinyl pyrrolidone) nanoparticles in 1N NaOH.

FIG. 8 is a representation of a backbone polymer with divinylcitrate cross-linkers according to one embodiment of the present invention.

FIGS. 9A-9D illustrate: (A) the synthesis of AHM cross linked amine functionalized nanoparticles encapsulating Gemcitabine; (B) the synthesis of amine functionalized AHM-nanoparticles encapsulating SPIO and gemcitabine; (C) the synthesis of amine functionalized AHM nanoparticles encapsulating photofrin; (D) the synthesis of amine functionalized AHM nanoparticles encapsulating photofrin and SPIO.

FIG. 10 illustrates the synthesis of F3 targeted nanoparticles encapsulating SPIO and Gemcitabine.

FIGS. 11a-11b illustrate the synthesis of F3 peptide conjugated nanoparticles encapsulating SPIO and gemcitabine.

FIG. 12 illustrates the synthesis of F3-C peptide conjugated nanoparticles encapsulating photofrin.

FIG. 13 illustrates the synthesis of C-F3 targeted nanoparticles encapsulating Gemcitabine and SPIO.

FIGS. 14a (4 hour incubation) and 14b (5 second incubation) show results of tumor cell kill using photodynarnic therapy in conjunction with targeted and non-targeted nanoparticles according to one embodiment of the present invention.

FIG. 15 shows ADC over maps for tumors treated using targeted and non-targeted photofrin-containing nanoparticles.

FIG. 16 shows a bar graph of the average % peak increase in tumor values following photodynamic therapy of the various groups.

FIG. 17 shows Kaplan-Meier survival statistics for rats treated with photodynamic therapy using nanoparticles according to one embodiment of the present invention.

FIG. 18 shows ADC overlay maps for radiosensitization studies performed using nanoparticles according to one embodiment of the present invention.

### DETAILED DESCRIPTION OF THE INVENTION

Suitable embodiments of the present invention are now described. While specific configurations and arrangements are discussed, it should be understood that this is done for illustrative purposes only. A person skilled in the relevant art will recognize that other configurations and arrangements can be used without departing from the spirit and scope of the invention.

The present invention provides for polymeric nanoparticles (referred to interchangeably herein as “nanoparticle(s)”) comprising a backbone polymer and a polymeric cross-linker that links two or more of the backbone polymers. Suitably, the nanoparticles of the present invention are used for drug and agent delivery, as well as for disease diagnosis and medical imaging in human and animal patients. The nanoparticles of the present invention can also be used in other applications such as chemical or biological reactions where a reservoir or depot is required.

As used herein, the term “nanoparticle” refers to particles between about 10 and about 1000 nm in diameter. In suitable embodiments, the diameter of the nanoparticles of the present invention will be less than about 200 nm in diameter, and more suitably less than about 100 nm in diameter. In certain such embodiments, the nanoparticles of the present invention will be between about 10 and about 200 nm, between about 30 and about 100 nm, or between about 40 and about 80 nm in diameter. As used herein, when referring to any numerical value, “about” means a value of ±10% of the stated value (e.g. “about 100 nm encompasses a range of diameters from 90 nm to 110 nm, inclusive).

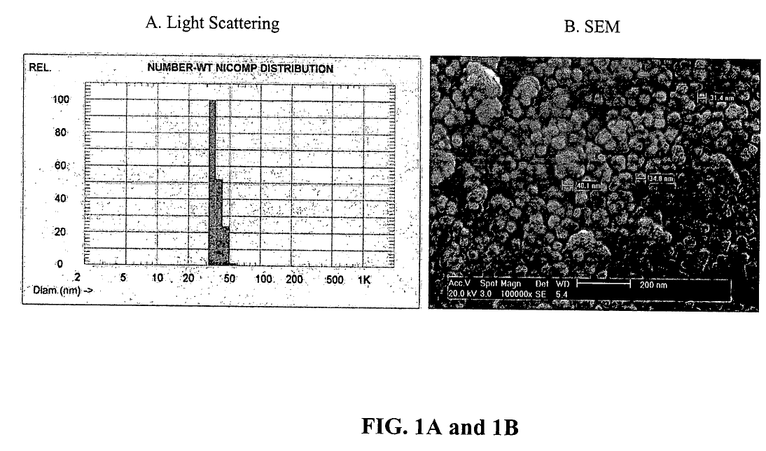

FIG. 1A shows multi angle light scattering size distribution, and FIG. 1B shows a scanning electron micrograph of nanoparticles of the present invention (amine-functionalized, 20% AHM nanoparticles encapsulating Photofrin and SPIO) demonstrating their fairly uniform size distribution and diameter. The small size of the nanoparticles of the present invention will allow them to evade capture by the RES, as well as extravasate from the vasculature, specifically in diseased areas such as the leaky vasculature of solid tumors.

TABLE 1
Light scattering particle size data (diameter) of various
nanoparticles according to the present invention.

| | Encapsulated Drug | Particle size, nm |
|---|---|---|
| Functionalization | (Approximate Amount) | (Diameter) |
| amine | Photofrin, 10 mg | 58.3 |
| amine | Photofrin, 15 mg | 52.6 |
| F3 | Gemcitabine, 20 mg | 67.8 |
| F3 | Gemcitabine, 40 mg | 60.7 |
| F3 | Photofrin, 10 mg | 59.7 |
| F3 | Photofrin, 15 mg | 68.9 |

Suitable backbone polymers for use in the nanoparticles of the present invention include, but are not limited to, poly(acrylamide), poly(2-hydroxyethyl methacrylate), poly(glycerol monomethacrylate), poly(acrylic acid), poly((aminoalkyl)methacrylamides), (e.g., poly(3-(aminopropyl) methacrylamide)poly(aminomethyl)methacrylamide), poly(aminoethyl) methacrylamide, poly(aminobutyl)methacrylamide, etc.), poly(sodium acrylate), and poly(vinyl pyrrolidone). In certain such suitable embodiments of the present invention, poly(acrylamide) is used as a backbone polymer. In other such embodiments, the backbone is poly(vinyl pyrrolidone). The term “backbone polymer” as used herein refers to the polymer units that make up the linear structure of the primary polymer component of the nanoparticles. In other embodiments, the backbone polymer can comprise mixtures of different polymers. For example, a mixture of poly(acrylamide) and poly(3-(aminopropyl)methacrylamide) (or suitable variants thereof) or a mixture of poly(acrylamide) and poly(acrylic acid).

The nanoparticles of the present invention also comprise a cross-linker that forms links between two or more of the backbone polymers. Suitable polymeric cross-linkers for use in the present invention include, but are not limited to glycerol(bis)acrylate (GBA), 3-(acryloyloxy)-2-hydroxypropyl methacrylate (AHM), ethylene glycol diacrylate, glycerol dimethacrylate, divinyl citrate and mixtures thereof.

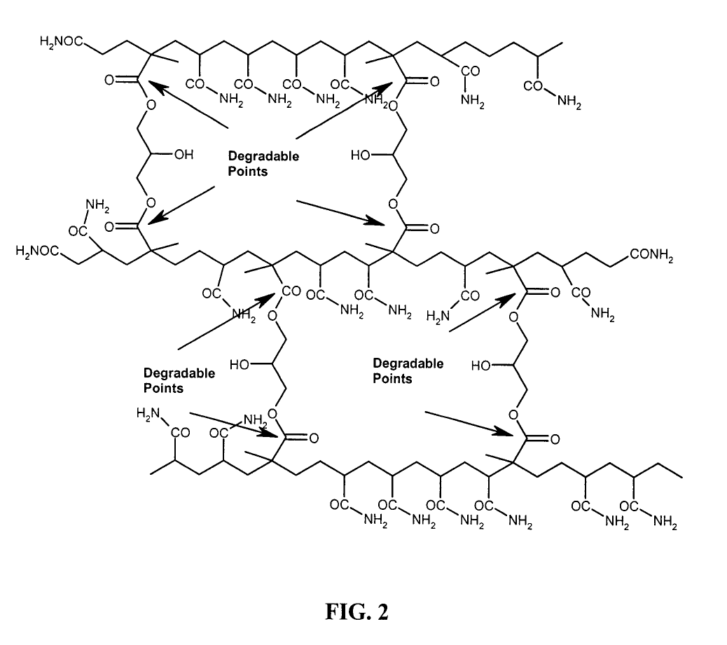

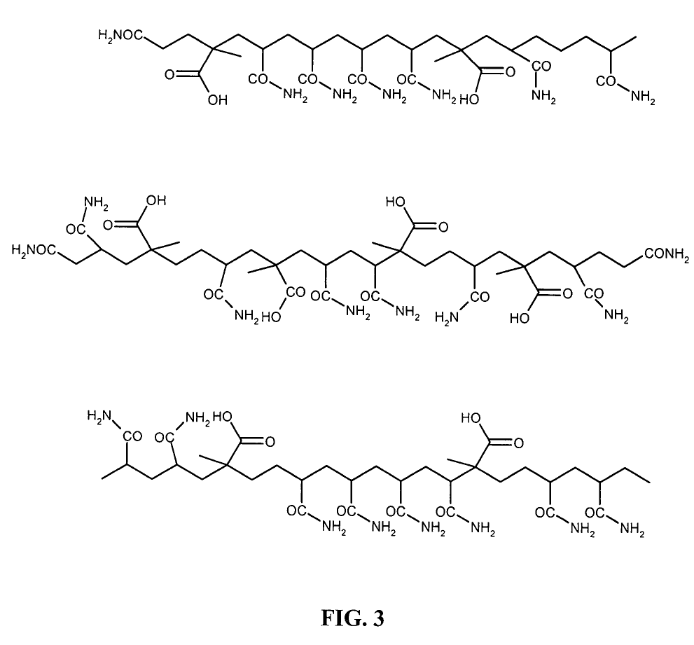

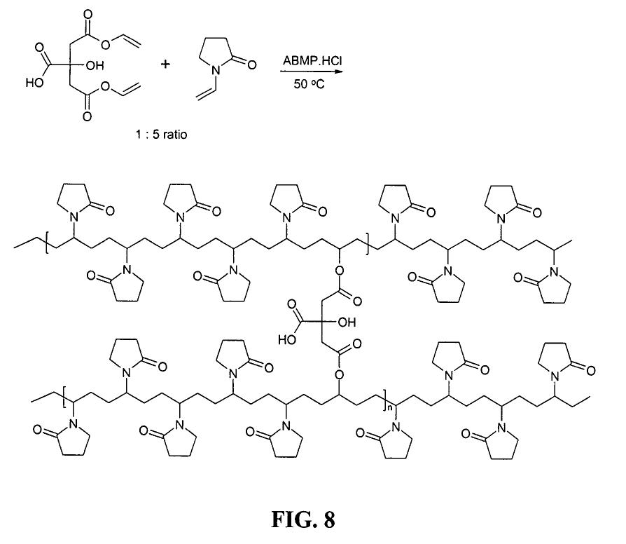

In suitable embodiments of the present invention when the backbone polymer is poly(acrylamide), the polymeric cross-linker is glycerol(bis) acrylate. One such embodiment is represented in FIG. 2 showing a poly(acrylamide) backbone cross-linked with glycerol(bis)acrylate. In other suitable embodiments of the present invention when the backbone polymer is poly(vinyl pyrrolidone), the polymeric cross-linker is divinyl citrate. FIG. 8 represents such an embodiment, showing a backbone polymer comprised of poly(vinyl pyrrolidone) cross-linked via 1,5-divinyl citrate. Other suitable embodiments of the present invention include, but are not limited to, where the backbone polymer is poly(acrylamide) and the polymeric cross-linker is 3-(acryloyloxy)-2-hydroxypropyl methacrylate, where the backbone polymer is a mixture of poly(acrylamide) and poly(3-(aminopropyl)methacrylamide) (or suitable variants) and the polymeric cross-linker is 3-(acryloyloxy)-2-hydroxypropyl methacrylate, where the backbone polymer is a mixture of poly(acrylamide) and poly(acrylic acid) and the polymeric cross-linker is 3-(acryloyloxy)-2-hydroxypropyl methacrylate, where the backbone polymer is a mixture of poly(acrylamide) and poly(3-(aminopropyl)methacrylamide) and the polymeric cross-linker is glycerol(bis)acrylate, and where the backbone polymer is a mixture of poly(acrylamide) and poly(acrylic acid) and the polymeric cross-linker is glycerol(bis)acrylate.

The polymeric nanoparticles of the present invention are prepared so as to be degradable, and suitably, to be biodegradable. The term “biodegradable” as used herein refers to both enzymatic and non-enzymatic breakdown or degradation of the polymeric structure. The polymeric cross-linkers utilized in the present invention provide specific degradation points, as shown in FIG. 2, where breakdown of the polymeric cross-linker may occur. Suitably, these degradation points will be carboxylic acid ester groups, though other biodegradable groups can be used in accordance with the present invention as can be determined by the ordinarily skilled artisan. When the nanoparticles of the present invention come in contact with the proteins, enzymes and hydrolyzing chemicals found in blood and other biological fluids, the polymeric cross-linkers are broken down. This degradation creates linear polymeric end products that can be readily excreted from the body. The degradation also provides for a method via which encapsulated contents, such as drugs or other agents, can be released at a site within the body. The rate of degradation and rate of release of encapsulated contents from the nanoparticles can be controlled through the selection of a specific combination of polymeric backbones and cross-linkers with appropriate calibration of the ratio of cross-linker(s) to backbone polymer(s). Varying the amount of cross-linker(s) (e.g. 5%, 10%, 15%, 20%, 25%, or 30%) relative to backbone monomer(s) (i.e., the density of the cross-linker relative to the backbone monomers) will allow for tailoring of the release rate of the encapsulated agent.

In other suitable embodiments of the present invention, the nanoparticles can comprise functionalized surface groups. Certain such functionalized surface groups include, but are not limited to, amine groups (e.g., primary or secondary amines), hydroxyl groups, thiolate groups and acidic groups (e.g., carboxylic acid groups or sulfonic acid groups). Such functional groups allow the attachment of targeting molecules to the surface of the nanoparticles for enhanced site-specific delivery of the nanoparticles. Attachment can be direct or through the use of a bifunctional linker such as 4-(maleimidomethyl)-1-cyclohexanecarboxylic acid N-hydroxysuccinimide ester (SMCC). Such targeting molecules include, but are not limited to antibody molecules, such as Herceptin® and Rituxan®, growth receptor ligands (e.g VEGF, erb-B2), peptides and other targeting molecules (e.g., haptens) known to those skilled in the art. Suitable targeting molecules include the F3 peptide (KDEPQRRSARLSAKPAPPKPEPKPKKAPAKK (SEQ ID NO: 1)), the F3-C peptide (KDEPQRRSARLSAKPAPPKPEPKPKKAPAKKC (SEQ ID NO: 2)) and the C-F3 peptide (CKDEPQRRSARLSAKPAPPKPEPKPKKAPAKK (SEQ ID NO:3)). See, e.g., Ruoslahti, E., et al., J. Cell Biol. 163:871-878 (2003). Drug molecules or imaging agents can also be attached to the functionalized molecules on the surface of the nanoparticles directly or through the use of an appropriate linker.

The nanoparticles of the present invention can also further comprise polymeric coatings on their surface that create a steric barrier to the approach of opsonins and other biological proteins, thereby prolonging blood circulation time. Such polymer coatings include poly(ethylene glycol) (PEG), suitably 500-5000 molecular weight, grafted to the surface. These PEG molecules can further comprise targeting molecules attached to their ends that facilitate delivery of the nanoparticles.

In suitable embodiments of the present invention, the nanoparticles can comprise one or more water-soluble, or water-insoluble agents, encapsulated inside. In addition, a water-soluble or water-insoluble agent can be attached to the surface nanoparticle via methods disclosed herein and well known in the art.

Suitable water-soluble and water-insoluble agents that can be encapsulated within the interior of the nanoparticles include small organic molecule drugs such as chemotherapeutic agents, including, but not limited to, alkylating agents such as busulfan, cis-platin, mitomycin C, and carboplatin; antimitotic agents such as colchicine, vinblastine, paclitaxel, and docetaxel; topoisomerase I inhibitors such as camptothecin and topotecan; topoisomerase II inhibitors such as doxorubicin and etoposide; RNA/DNA antimetabolites such as 5-azacytidine, 5-fluorouracil and methotrexate; DNA antimetabolites such as 5-fluoro-2′-deoxy-uridine, ara-C, gemcitabine (2′,2′-difluorodeoxycytidine) (Gemzar®), hydroxyurea and thioguanine; antibodies such as Herceptin® and Rituxan®; therapeutic vaccines; antiangionetics; kinase inhibitors; as well as other known chemotherapeutics such as photofrin, melphalan, chlorambucil, cyclophosamide, ifosfamide, vincristine, mitoguazone, epirubicin, aclarubicin, bleomycin, mitoxantrone, elliptinium, fludarabine, octreotide, retinoic acid, tamoxifen and alanosine. The agents can also be covalently attached to the surface of the nanoparticle or the polymer backbone.

The nanoparticles of the present invention can also be used to encapsulate DNA, RNA, siRNA, proteins and other polymers. Suitable such proteins and polymers will be less than about 10 nm in size.

In certain embodiments of the present invention, the nanoparticles can be used to encapsulate one or more fluorescent dyes, such as carboxyfluorescein, ruthenium, and rhodamine; one or more radioisotopes; one or more Magnetic Resonance Imaging (MRI) contrast agents, such as iron oxide (e.g., superparamagnetic iron oxide (SPIO)); or one or more contrast agents. For example, Gadolinium (Gd) complexes or Gadolinium chelates (e.g., Gadolinium DTPA, Gd DOTA, Gadomer-17) and the polymers of such materials can be incorporated. Such contrast agents can be either chemically attached to the surface of the nanoparticles or encapsulated. Similarly, polyiodinated compounds can be incorporated in the nanoparticles. In addition, ¹⁰B enriched compounds can be incorporated in the degradable nanoparticles for BNCT studies. These agents can be used to allow for identification the nanoparticles in vivo in human and animal patients. Gadolinium-complexes can also be used in for Neutron Capture Therapy (Gd-NCT) applications. See e.g., Matsumura, A., et al., Anticancer Res. 23:2451-2456 (2003), Shikata F., et al., Eur. J. Pharm. Biopharm. 53:57-63 (2002) and Tokumitsu H., et al., Cancer Lett. 150:177-182 (2000).

In other suitable embodiments, the nanoparticles of the present invention can comprise two or more different agents from the groups described throughout. For example, the nanoparticles of the present invention can incorporate a combination of agents including, a chemotherapeutic agent, a radioisotope, and an imaging or contrast agent encapsulated within the same nanoparticle. Suitably, this nanoparticle can then be surface modified to incorporate a PEG coating and/or an antibody or other targeting molecule on its surface.

The polymeric nanoparticles of the present invention can also encapsulate degradable polymers, including, but not limited to, polyesters such as poly(lacti-glycolic acid) (PLGA), polysorbitol, polysorbitol-adipate, polymannitol, poly amino acids such as polyaspartic acid, polylysine and polyglutamic acid. These polymers can be used to further control the release or retention of a drug, imaging agent or other encapsulated substance. In suitable embodiments, the degradable polymers are co-encapsulated with a second agent.

The polymeric nanoparticles can be used for various methods of treatment and/or diagnosis in human and animal patients. In certain embodiments, the present invention provides methods of treating a tumor in a mammalian patient comprising: administering to the patient a polymeric nanoparticle according to the present invention, wherein the polymeric nanoparticle encapsulates one or more cancer chemotherapeutic agents. Suitable chemotherapeutic agents include those known in the art and disclosed throughout, and include gemcitabine and photofrin. In suitable embodiments, the nanoparticle can further encapsulate an imaging agent. Imaging agents that can be encapsulated are well known in the art and include those disclosed throughout, such as iron oxide. The present invention also provides methods of imaging the polymeric nanoparticles which encapsulate imaging agents.

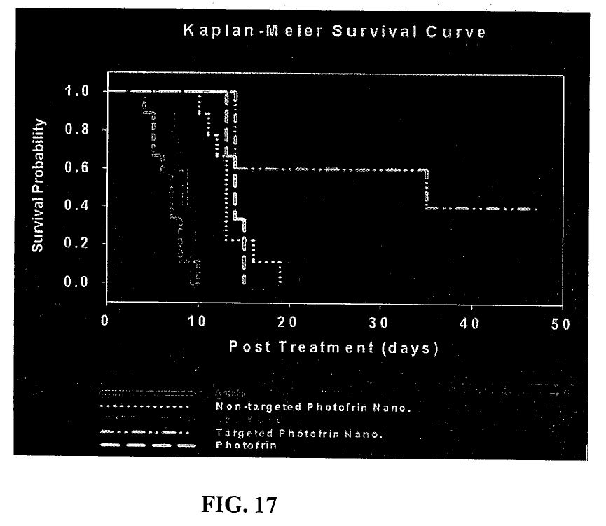

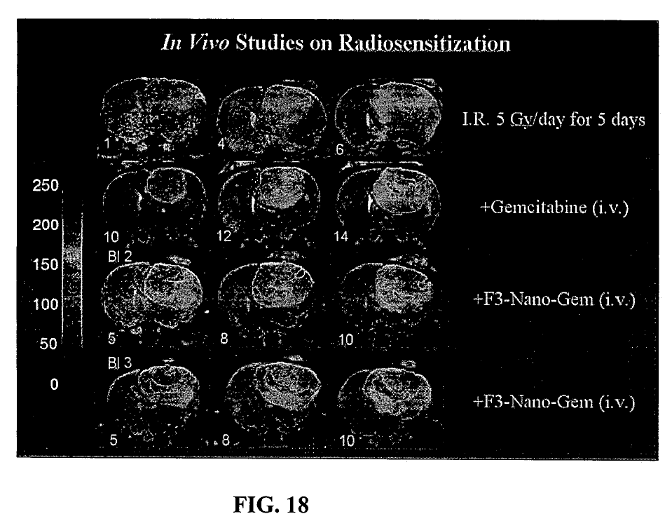

In other embodiments, the polymeric nanoparticles can be used to treat tumors by encapsulating a photodynamic therapeutic drug within the targeted nanoparticle. In certain embodiments, the present investigation provides methods to encapsulate photofrin, a photodynamic therapeutic agent, in a targeted nanoparticle and evaluating the efficacy of the therapy by diffusion MRI. FIG. 17 shows that the survival rate of brain tumor bearing animals treated with F3 targeted photodynamic therapeutic nanoparticles.

In other embodiments, the polymeric nanoparticles can be used to deliver radiation-sensitizing agents to tumors. In such embodiments, polymeric nanoparticles encapsulating one or more radiation-sensitizing agents are administered to a patient in need of such treatment and ionizing radiation is administered to the patient. Suitably, the radiation-sensitizing agents are released from the nanoparticles at the tumor site such that the ionizing radiation can act upon the agents at the tumor site. Radiation-sensitizing agents include any agent that increases the sensitivity of a tumor to ionizing radiation and include, but are not limited to, gemcitabine, paclitaxel, carboplatin, and other such compounds. In other embodiments, an imaging agent, such as those described herein, can be co-encapsulated with the radiation-sensitizing agent (or attached to the surface of the nanoparticle) to allow for imaging of the nanoparticles prior to and/or during radiation treatment. The nanoparticles can also comprise a targeting molecule, such as those described herein, to allow for targeting of the nanoparticles to the tumor tissue.

The present invention also provides methods of producing a nanoparticle comprising (a) forming a solution of polymeric monomers and a polymeric cross-linker, (b) initiating polymerization to generate a solid particle comprising a polymeric backbone of the polymeric monomer cross-linked with the polymeric cross-linker; and (c) removing the solid particle from solution.

In suitable embodiments of the methods of the present invention, the monomers used to generate the polymeric backbone include, but are not limited to, acrylamide, 2-hydroxyethyl methacrylate, glycerol monomethacrylate, acrylic acid, sodium acrylate, vinyl pyrrolidone, and (aminoalkyl)methacrylamides, e.g., 3-(aminopropyl)methacrylamide, (aminomethyl)methacrylamide, (aminoethyl)methacrylamide, (aminobutyl) methacrylamide, etc., as well as combinations and mixtures of these polymers. For example, a mixture of poly(acrylamide) and poly(3-(aminopropyl)methacrylamide) or a mixture of poly(acrylamide) and poly(acrylic acid). The cross-linkers used in the methods of the present invention include, but are not limited to, glycerol(bis)acrylate, 3-(acryloyloxy)-2-hydroxypropyl methacrylate, ethylene glycol diacrylate, glycerol dimethacrylate, and divinyl citrate, as well as combinations and mixtures of these cross linkers. Suitable embodiments include, but are not limited to, a poly(acrylamide) backbone cross-linked with glycerol(bis)acrylate, a poly(vinyl pyrrolidone) backbone and a divinyl citrate cross-linker, and a backbone polymer comprised of poly(vinyl pyrrolidone) cross-linked via 1,5-divinyl citrate. Other suitable embodiments of the present invention include, but are not limited to, where the backbone polymer is poly(acrylamide) and the polymeric cross-linker is 3-(acryloyloxy)-2-hydroxypropyl methacrylate, where the backbone polymer is a mixture of poly(acrylamide) and poly(3-(aminopropyl)methacrylamide) and the polymeric cross-linker is 3-(acryloyloxy)-2-hydroxypropyl methacrylate, where the backbone polymer is a mixture of poly(acrylamide) and poly(acrylic acid) and the polymeric cross-linker is 3-(acryloyloxy)-2-hydroxypropyl methacrylate, where the backbone polymer is a mixture of poly(acrylamide) and poly(3-(aminopropyl)methacrylamide) and the polymeric cross-linker is glycerol(bis)acrylate, and where the backbone polymer is a mixture of poly(acrylamide) and poly(acrylic acid) and the polymeric cross-linker is glycerol(bis)acrylate.

In suitable embodiments of the present invention, the solution that is formed of polymeric monomers and cross-linkers is a water-based solution, suitably a sodium phosphate buffer, though non-water based solutions can also be used, such as, but not limited to, acetonitrile, dimethylformamide and methanol. Polymerization can be initiated by any initiation protocol known to those skilled in the art. Suitably, ammonium persulfate and N,N,N,N-tetra methyl ethylenediamine (TEMED) are used to initiate polymerization. Polymerization generates cross-links between the polymeric backbone comprised of monomer units, and the cross-linking molecules, to generate a cross-linked polymer network, as shown in FIGS. 2 and 8. In certain embodiments, the methods of present invention will comprise the use of surfactants or similar substances to aid in formation and sizing of the polymeric nanoparticles. Suitable surfactants include, but are not limited to Brij 30 and dioctyl sulfosuccinate (AOT).

Following polymerization and nanoparticle formation, the solid particles that are formed are filtered and washed, and then dried. The solid particles can then be suspended in a water-based solution, and filtered or extruded through one or more filters with an appropriate pore size, to generate nanoparticles that are less than about 200 nm in diameter, suitably less than about 100 nm in diameter.

In suitable embodiments of the present invention, one or more water-soluble, or water-insoluble agents can be added to the solution of polymer monomer units and cross-linkers prior to initiation of polymerization. Following polymerization, a solid particle is generated that has the agents encapsulated within its interior. Suitable agents for encapsulation are described throughout the present specification and well known by those skilled in the art.

The methods of the present invention can further comprise the generation of a functional group on the surface of the nanoparticle. Suitably this functional group can be an amine group (e.g., primary or secondary amines), acid group (e.g., carboxylic acid or sulfonic acid), alcohol, or thiol group of another monomer that can be added to the solution prior to polymerization (e.g. N-(3-aminopropyl)methacrylamide). In other suitable embodiments, the methods of the present invention can comprise the addition of a PEG or antibody molecule to the surface of nanoparticle.

The following represent non-limiting, exemplary combinations of monomers and cross-linkers that can be used to create nanoparticles according to the methods of present invention:
a) Acrylamide and glycerol dimethacrylate 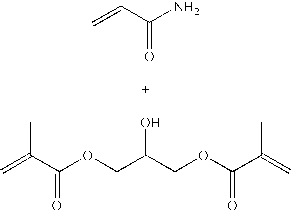
b) Acrylamide and 3-(acryloyloxy)-2-hydroxypropyl methacrylate 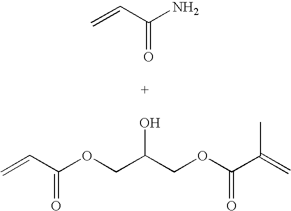
c) Acrylamide and ethylene glycol diacrylate 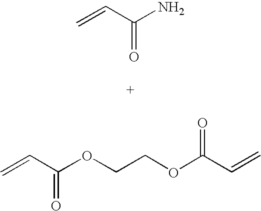
d) 2-Hydroxyethyl methacrylate and Glycerol dimethacrylate 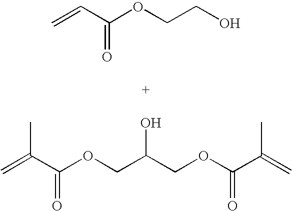
e) Glycerol monomethacrylate and glycerol dimethacrylate 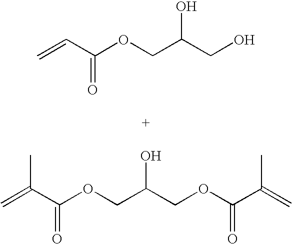
f) Acrylic acid and glycerol dimethacrylate 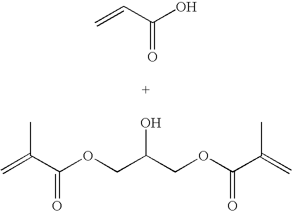
g) Sodium acrylate and glycerol dimethacrylate

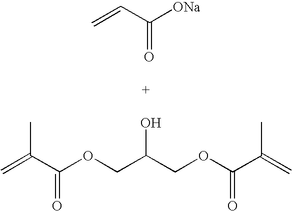

The present invention also provides polymeric nanoparticles comprising a backbone polymer selected from the group consisting of poly(acrylamide), poly(acrylic acid), poly(3-(aminopropyl)methacrylamide) and mixtures thereof, cross-linked with; about 10% glycerol(bis)acrylate cross-linker; and a functionalized surface group conjugated to a peptide selected from the group consisting of SEQ ID NO: 1, SEQ ID NO: 2 and SEQ ID NO: 3, wherein the polymeric nanoparticle encapsulates iron oxide. In suitable embodiments, the polymeric nanoparticles can comprise about 20% glycerol(bis)acrylate cross-linker. In other embodiments, the nanoparticles can comprise about 10% or about 20% 3-(acryloyloxy)-2-hydroxypropyl methacrylate cross-linker. The nanoparticles can also further encapsulate gemcitabine.

In another embodiment, the present invention provides polymeric nanoparticles produced by the process comprising: forming a solution of polymeric monomers and cross-linkers, the monomers selected from the group consisting of acrylamide, 3-(aminopropyl)methacrylamide, acrylic acid, and mixtures thereof; the cross-linkers selected from the group consisting of glycerol(bis)acrylate, 3-(acryloyloxy)-2-hydroxypropyl methacrylate and mixtures thereof; optionally adding a functionalized monomer; adding iron oxide; initiating polymerization to generate a solid particle, the particle comprising a polymeric backbone of the polymeric monomers cross-linked with the polymeric cross-linker; conjugating a peptide selected from the group consisting of SEQ ID NO: 1, SEQ ID NO: 2 and SEQ ID NO: 3 to a functionalized surface group on the nanoparticle; and removing the nanoparticle from solution, wherein the cross-linker density is about 10% relative to the polymeric backbone. In suitable embodiments, gemcitabine can be added prior to initiation of polymerization. In other embodiments, the cross-linker density can be about 20% relative to the polymeric backbone.

The nanoparticles of the present invention can suitably be used for delivery of agents to a diseased site, and in the diagnosis or imaging of a specific tissue or site. In suitable embodiments, the nanoparticles can encapsulate several agents, including chemotherapeutic agents, contrast agents, and radioisotopes, within the same nanoparticle. These nanoparticles can further comprise targeting molecules on their surface. The nanoparticles of the present invention are especially useful for the treatment, diagnosis and imaging of solid tumors, including, but not limited to, cancers of the brain, breast, limbs, lung, heart, and gut.

It will be readily apparent to one of ordinary skill in the relevant arts that other suitable modifications and adaptations to the methods and applications described herein may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

### Example 1

Synthesis of Blank Polymeric Nanoparticles

The following synthesis is on a 2 g scale and it can be extended from multi gram to kilogram level.

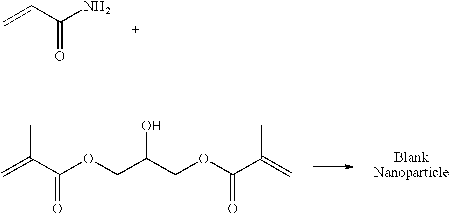

A clean 20 ml glass vial was charged with acrylamide (1.2 g) and 4 ml of sodium phosphate buffer (10 mM, pH 7.3). The suspension was sonicated for 2 min to obtain a clear solution. Glycerol(bis)acrylate (0.53 g) was added to the reaction mixture and sonicated for an additional 5 min. The resulting slightly turbid monomer solution was added to a 250 ml round bottom flask containing an argon-purged, well stirred solution of dioctyl sulfosuccinate (AOT) (3.2 g) and Brij 30 (6.4 ml) in hexanes (90 ml). After a 10 min stirring under an argon blanket at room temperature, the reaction mixture was treated with freshly prepared aqueous ammonium persulfate (65 μl, 10%) and N,N,N,N-tetra methyl-ethylenediamine (TEMED) (85 μl) to initiate polymerization. The reaction mixture was gently stirred at room temperature overnight to ensure complete polymerization.

The reaction mixture was then concentrated to a thick residue and re-suspended in ethanol (100 ml). The precipitated particles were filtered and thoroughly washed with ethanol (5×160 ml) in an Amicon stirred cell equipped with a Biomax filter membrane (500 KDa, filtration pressure 10 psi, nitrogen). The solid material was transferred onto a Whatman filter paper, gently crushed into a fine powder, and subjected to air-drying until a constant weight was observed (3-4 hrs). (Typical yield around 100%.) The product (white free flowing powder) can be stored at 4° C. for extended periods of time.

The product was suspended in water (20 mg/ml) and sonicated to get a homogenous solution. The solution was transferred into an amicon stirred cell equipped with a Biomax (500 Kda) filter membrane and thoroughly washed with water (5×150 ml). The concentrated sample (˜50 mg/ml) was passed through 0.45μ and 0.2μ filters and stored at 4° C. until further use.

### Example 2

Synthesis of Amine Functionalized Degradable Nanoparticles.

The synthesis is on a 4 g scale and it can be extended to kilogram level.

The monomer solution was prepared by adding acrylamide (2.4 g), N-(3-aminopropyl)-methacrylamide hydrochloride (0.4 g) and glycerol(bis) acrylate (1.06 g) to sodium phosphate buffer (8 ml, 10 mM, pH 7.3). The slightly turbid mixture was sonicated for 10 min and added to a solution containing AOT (6.4 g) and Brij 30 (12.8 ml) in argon purged hexanes (180 mL). After a 10 min stirring at ambient temperature, the polymerization reaction was initiated by treating with a freshly prepared aqueous ammonium per sulfate (130 μl, 10%) and TEMED (170 μl). The reaction mixture was stirred over night under an argon atmosphere.

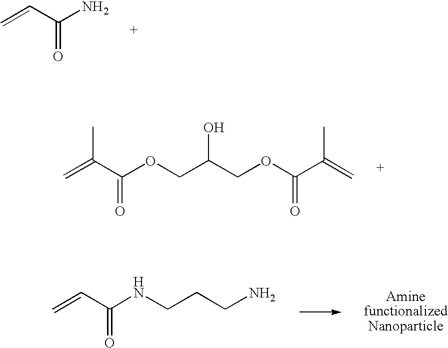

Hexane was removed under reduced pressure and the residue was treated with ethanol (150 ml). The precipitated nanoparticle solution was transferred into an amicon stirred cell (200 ml) equipped with a 500 KDa Biomax filter membrane (filtration pressure 10 psi, nitrogen), washed thoroughly with ethanol (5×160 ml) and air dried. The solid material was gently crushed to a fine free flowing white powder (yield 100%). The product can be stored at 4° C. for extended periods of time.

The product was suspended in water (20 mg/ml) and sonicated to give a homogenous solution. The solution was transferred into an amicon stirred cell equipped with a Biomax (500 Kda) filter membrane and thoroughly washed with water (5×150 ml). The concentrated sample (˜50 mg/ml) was passed through 0.45μ and 0.2μ filters and stored at 4° C. until further use.

### Example 3

Synthesis of Nanoparticles Encapsulating Super Paramagnetic Iron Oxide (SPIO).

The following synthesis is on a 2 g scale and it can be extended from multi gram to kilogram level.

A 20 ml glass vial was charged with acrylamide (1.2 g) and 2 ml of sodium phosphate buffer (10 mM, pH 7.3). The suspension was sonicated for 2 min to obtain a clear solution. Glycerol(bis)acrylate (0.53 g) was added to the reaction mixture and sonicated for an additional 5 min. The resulting slightly turbid monomer solution was treated with Iron oxide solution (180 mg) and the deep dark mixture was sonicated for 10 min.

A 250 ml round bottom flask equipped with a mechanical stirrer was charged with AOT (3.2 g) and Brij 30 (6.4 ml) in argon purged hexanes (90 ml). The clear solution was treated with the above iron oxide monomer solution with stirring. After a 10 min mechanical stirring (high speed) under an argon blanket at room temperature, the polymerization was initiated by treating reaction mixture with a freshly prepared aqueous ammonium per sulfate (65 μl, 10%) and N,N,N,N-tetramethylethylenediamine (TEMED) (85 μl). The reaction mixture was stirred at room temperature overnight.

The solvent was removed under reduced pressure to obtain a black thick residue. The resulting thick residue was re-suspended in ethanol (100 mL) and the precipitated nanoparticles were filtered and thoroughly washed with ethanol (5×160 ml) in an Amicon stirred cell (200 ml) equipped with a Biomax filter membrane (500 Kda, filtration pressure 10 psi, nitrogen). The solid material was transferred onto a Whatman filter paper, gently crushed into a fine powder and subjected to air-drying until a constant weight was observed (3-4 hrs). The product (Black free flowing powder) can be stored at 4° C. for extended periods of time.

The product was suspended in water (20 mg/ml) and sonicated to give a homogenous solution. The solution was transferred into an amicon stirred cell equipped with a Biomax (500 Kda) filter membrane and thoroughly washed with water (5×150 ml). The concentrated sample (˜50 mg/ml) was passed through 0.45μ and 0.2μ filters and stored at 4° C. until further use.

### Example 4

Synthesis of Nanoparticles Encapsulating Ruthenium Dye.

The following synthesis is on a 2 g scale and it can be extended from multi gram to kilogram level.

A clean 20 ml glass vial was charged with acrylamide (1.2 g) and 4 ml of sodium phosphate buffer (10 mM, pH 7.3). The suspension was sonicated for 2 min to obtain a clear solution. Glycerol(bis)acrylate (0.53 g) was added to the reaction mixture and sonicated for an additional 5 min. Ruthenium dye (5 mg, Ru(dpp)(SO₃Na)₂)₃ disulfonated 4,7-diphenyl-1,10-phenantroline Ruthenium) was added and the mixture was sonicated for an additional 5 min. The resulting slightly turbid monomer solution was added to a 250 ml round bottom flask containing an argon-purged, well stirred solution of dioctyl sulfosuccinate (3.2 g) and Brij 30 (6.4 ml) in hexanes (90 ml). After a 10 min stirring under an argon blanket at room temperature, the reaction mixture was treated with freshly prepared aqueous ammonium per sulfate (65 μl, 10%) and TEMED (85 μl) to initiate the polymerization. The reaction mixture was gently stirred at room temperature overnight to ensure complete polymerization.

The reaction mixture was concentrated to a thick residue and re-suspended in ethanol (100 ml). The precipitated particles were filtered and thoroughly washed with ethanol (5×160 ml) in an Amicon stirred cell equipped with a Biomax filter membrane (500 Kda, filtration pressure 10 psi, nitrogen). The solid material was transferred onto a Whatman filter paper, gently crushed into a fine powder and subjected to air-drying until a constant weight was observed (3-4 hrs). The product (free flowing light pink powder) was stored at 4° C. for an extended period. The yield of the product was 100%.

The product was suspended in water (20 mg/ml) and sonicated to get a homogenous solution. The solution was transferred into an amicon stirred cell equipped with a Biomax (500 Kda) filter membrane and thoroughly washed with water (5×150 ml). The concentrated sample (˜50 mg/ml) was passed through 0.45μ and 0.2μ filters and stored at 4° C. until further use.

### Example 5

Synthesis of 10% Glycerol(Bis)Acrylate (Gba) Cross-Linked Blank Polymeric Nanoparticles.

A clean 20 ml glass vial was charged with acrylamide (1.2 g) and 4 ml of sodium phosphate buffer (10 mM, pH 7.3). The suspension was sonicated for 2 min to obtain a clear solution. GBA (0.453 g, 10% cross-links) was added to the reaction mixture and sonicated for an additional 5 min. The resulting slightly turbid monomer solution was added to a 250 ml round bottom flask containing an argon-purged, well stirred solution of dioctyl sulfosuccinate (AOT) (3.2 g) and Brij 30 (6.4 ml) in hexanes (90 ml). After a 10 min stirring under an argon blanket at room temperature, the reaction mixture was treated with freshly prepared aqueous ammonium persulfate (65 μl, 10%) and TEMED (85 μl) to initiate polymerization. The reaction mixture was gently stirred at room temperature overnight to ensure complete polymerization.

The reaction mixture was then concentrated to a thick residue and re-suspended in ethanol (100 ml). The precipitated particles were filtered and thoroughly washed with ethanol (5×160 ml) in an Amicon stirred cell equipped with a Biomax filter membrane (500 KDa, filtration pressure 10 psi, nitrogen). The solid material was transferred onto a Whatman filter paper, gently crushed into a fine powder, and subjected to air-drying until a constant weight was observed (3-4 hrs, typical yield 100%) The product (1.87 g, white free flowing powder) was stored at 4° C. for extended periods of time.

The product was suspended in water (20 mg/ml) and sonicated to get a homogenous solution. The solution was transferred into an amicon stirred cell equipped with a Biomax (500 Kda) filter membrane and thoroughly washed with water (5×150 ml). The concentrated sample (˜50 mg/ml) was passed through 0.45μ and 0.2μ filters and stored at 4° C. until further use.

### Example 6

Synthesis of 15% GBA Cross-Linked Blank Polymeric Nanoparticles.

A clean 20 ml glass vial was charged with acrylamide (1.2 g) and 4 ml of sodium phosphate buffer (10 mM, pH 7.3). The suspension was sonicated for 2 min to obtain a clear solution. GBA (0.679 g, 15% cross-links) was added to the reaction mixture and sonicated for an additional 5 min. The resulting slightly turbid monomer solution was added to a 250 ml round bottom flask containing an argon-purged, well stirred solution of dioctyl sulfosuccinate (AOT) (3.2 g) and Brij 30 (6.4 ml) in hexanes (90 ml). After a 10 min stirring under an argon blanket at room temperature, the reaction mixture was treated with freshly prepared aqueous ammonium persulfate (65 μl, 10%) and TEMED (85 μl) to initiate polymerization. The reaction mixture was gently stirred at room temperature overnight to ensure complete polymerization.

The reaction mixture was then concentrated to a thick residue and re-suspended in ethanol (100 ml). The precipitated particles were filtered and thoroughly washed with ethanol (5×160 ml) in an Amicon stirred cell equipped with a Biomax filter membrane (500 KDa, filtration pressure 10 psi, nitrogen). The solid material was transferred onto a Whatman filter paper, gently crushed into a fine powder, and subjected to air-drying until a constant weight was observed (3-4 hrs). The product (2.2 g, white free flowing powder) was stored at 4° C. for extended periods of time.

The product was suspended in water (20 mg/ml) and sonicated to get a homogenous solution. The solution was transferred into an amicon stirred cell equipped with a Biomax (500 Kda) filter membrane and thoroughly washed with water (5×150 ml). The concentrated sample (˜50 mg/ml) was passed through 0.45μ and 0.2μ filters and stored at 4° C. until further use.

### Example 7

Synthesis of 20% GBA Cross-Linked Blank Polymeric Nanoparticles.

A clean 20 ml glass vial was charged with acrylamide (1.2 g) and 4 ml of sodium phosphate buffer (10 mM, pH 7.3). The suspension was sonicated for 2 min to obtain a clear solution. GBA (0.905 g, 20% cross-links) was added to the reaction mixture and sonicated for an additional 5 min. The resulting slightly turbid monomer solution was added to a 250 ml round bottom flask containing an argon-purged, well stirred solution of dioctyl sulfosuccinate (AOT) (3.2 g) and Brij 30 (6.4 ml) in hexanes (90 ml). After a 10 min stirring under an argon blanket at room temperature, the reaction mixture was treated with freshly prepared aqueous ammonium persulfate (65 μl, 10%) and TEMED (85 μl) to initiate polymerization. The reaction mixture was gently stirred at room temperature overnight to ensure complete polymerization.

The reaction mixture was then concentrated to a thick residue and re-suspended in ethanol (100 ml). The precipitated particles were filtered and thoroughly washed with ethanol (5×160 ml) in an Amicon stirred cell equipped with a Biomax filter membrane (500 KDa, filtration pressure 10 psi, nitrogen). The solid material was transferred onto a Whatman filter paper, gently crushed into a fine powder, and subjected to air-drying until a constant weight was observed (3-4 hrs). The product (2.34 g, white free flowing powder) was stored at 4° C. for extended periods of time.

The product was suspended in water (20 mg/ml) and sonicated to get a homogenous solution. The solution was transferred into an amicon stirred cell equipped with a Biomax (500 Kda) filter membrane and thoroughly washed with water (5×150 ml). The concentrated sample (˜50 mg/ml) was passed through 0.45μ and 0.2μ filters and stored at 4° C. until further use.

### Example 8

Synthesis of 25% GBA Cross-Linked Blank Polymeric Nanoparticles.

A clean 20 ml glass vial was charged with acrylamide (1.2 g) and 4 ml of sodium phosphate buffer (10 mM, pH 7.3). The suspension was sonicated for 2 min to obtain a clear solution. GBA (1.13 g, 25% cross-links) was added to the reaction mixture and sonicated for an additional 5 min. The resulting slightly turbid monomer solution was added to a 250 ml round bottom flask containing an argon-purged, well stirred solution of dioctyl sulfosuccinate (AOT) (3.2 g) and Brij 30 (6.4 ml) in hexanes (90 ml). After a 10 min stirring under an argon blanket at room temperature, the reaction mixture was treated with freshly prepared aqueous ammonium persulfate (65 μl, 10%) and TEMED (85 μl) to initiate polymerization. The reaction mixture was gently stirred at room temperature overnight to ensure complete polymerization.

The reaction mixture was then concentrated to a thick residue and re-suspended in ethanol (100 ml). The precipitated particles were filtered and thoroughly washed with ethanol (5×160 ml) in an Amicon stirred cell equipped with a Biomax filter membrane (500 KDa, filtration pressure 10 psi, nitrogen). The solid material was transferred onto a Whatman filter paper, gently crushed into a fine powder, and subjected to air-drying until a constant weight was observed (3-4 hrs). The product (3.12 g, white free flowing powder) was stored at 4° C. for extended periods of time.

The product was suspended in water (20 mg/ml) and sonicated to get a homogenous solution. The solution was transferred into an amicon stirred cell equipped with a Biomax (500 Kda) filter membrane and thoroughly washed with water (5×150 ml). The concentrated sample (˜50 mg/ml) was passed through 0.45μ and 0.2μ filters and stored at 4° C. until further use.

### Example 9

Synthesis of 30% GBA Cross-Linked Blank Polymeric Nanoparticles.

A clean 20 ml glass vial was charged with acrylamide (1.2 g) and 4 ml of sodium phosphate buffer (10 mM, pH 7.3). The suspension was sonicated for 2 min to obtain a clear solution. GBA (1.36 g, 30% cross-links) was added to the reaction mixture and sonicated for an additional 5 min. The resulting slightly turbid monomer solution was added to a 250 ml round bottom flask containing an argon-purged, well stirred solution of dioctyl sulfosuccinate (AOT) (3.2 g) and Brij 30 (6.4 ml) in hexanes (90 ml). After a 10 min stirring under an argon blanket at room temperature, the reaction mixture was treated with freshly prepared aqueous ammonium persulfate (65 μl, 10%) and TEMED (85 μl) to initiate polymerization. The reaction mixture was gently stirred at room temperature overnight to ensure complete polymerization.

The reaction mixture was then concentrated to a thick residue and re-suspended in ethanol (100 ml). The precipitated particles were filtered and thoroughly washed with ethanol (5×160 ml) in an Amicon stirred cell equipped with a Biomax filter membrane (500 KDa, filtration pressure 10 psi, nitrogen). The solid material was transferred onto a Whatman filter paper, gently crushed into a fine powder, and subjected to air-drying until a constant weight was observed (3-4 hrs). The product (2.72 g, white free flowing powder) was stored at 4° C. for extended periods of time.

The product was suspended in water (20 mg/ml) and sonicated to get a homogenous solution. The solution was transferred into an amicon stirred cell equipped with a Biomax (500 Kda) filter membrane and thoroughly washed with water (5×150 ml). The concentrated sample (˜50 mg/ml) was passed through 0.45μ and 0.2μ filters and stored at 4° C. until further use.

### Example 10

Synthesis of 10% 3-(acryloyloxy)-2-hydroxypropyl methacrylate (AHM) Nanoparticles Encapsulating Super Paramagnetic Iron Oxide (SPIO).

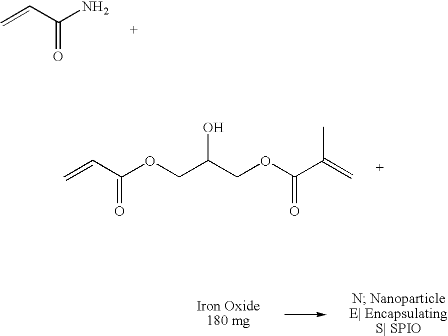

A clean 250 ml round bottom flask was charged with dioctylsulfosuccinate, disodium salt (3.2 g) and Brij 30 (6.4 ml) in argon purged hexanes (90 ml). The mixture was stirred at room temperature for 15 min to obtain a clear solution. A clean 20 ml glass vial was charged with acrylamide (1.2 g, 16.9 mmol) and 3 ml of sodium phosphate buffer (pH 7.3, 10 mM). After a brief vortex, the monomer solution was treated with AHM (0.362 g, 1.69 mmol, for 10% cross-links). The mixture was sonicated for 2 min and treated with iron oxide solution (180 mg, 1 ml). The resulting monomer solution was sonicated for 5 min and added to the hexane solution under argon atmosphere with stirring. After a 20 min vigorous stirring at room temperature, the polymerization was initiated with a freshly prepared 10% ammonium per sulfate (0.065 ml) and TEMED (0.085 ml) solution. The reaction mixture was stirred at room temperature overnight.

The reaction mixture was concentrated to a thick residue under reduced pressure and the residue was diluted with 100 ml of ethanol. After a brief sonication (2 min), the mixture was transferred into an amicon stirred cell equipped with a 500 KDa polyether sulfone membrane filter and thoroughly washed with ethanol (5×180 ml). The black solid material was dried and crushed gently to a fine powder. The product was stored at 4° C. until further use. The yield of the product was 1.9 g.

### Example 1

Synthesis of 15% AHM Nanoparticles Encapsulating SPIO.

A clean 250 ml round bottom flask was charged with dioctylsulfosuccinate, disodium salt (3.2 g) and Brij 30 (6.4 ml) in argon purged hexanes (90 ml). The mixture was stirred at room temperature for 15 min to obtain a clear solution. A clean 20 ml glass vial was charged with acrylamide (1.2 g, 16.9 mmol) and 3 ml of sodium phosphate buffer (pH 7.3, 10 mM). After a brief vortex, the monomer solution was treated with AHM (0.54 g, 2.5 mmol, for 15% cross-links). The mixture was sonicated for 2 min and treated with iron oxide solution (180 mg, 1 ml). The resulting monomer solution was sonicated for 5 min and added to the hexane solution under argon atmosphere with stirring. After a 20 min vigorous stirring at room temperature, the polymerization was initiated with a freshly prepared 10% ammonium per sulfate (0.065 ml) and TEMED (0.085 ml) solution. The reaction mixture was stirred at room temperature overnight.

The reaction mixture was concentrated to a thick residue under reduced pressure and the residue was diluted with 100 ml of ethanol. After a brief sonication (2 min), the mixture was transferred into an amicon stirred cell equipped with a 500 KDa polyether sulfone membrane filter and thoroughly washed with ethanol (5×180 ml). The black solid material was dried and crushed gently to a fine powder. The product was stored at 4° C. until further use. The yield of the product was 2.0 g.

### Example 12

Synthesis of 20% AHM Nanoparticles Encapsulating SPIO.

A clean 250 ml round bottom flask was charged with dioctylsulfosuccinate, disodium salt (3.2 g) and Brij 30 (6.4 ml) in argon purged hexanes (90 ml). The mixture was stirred at room temperature for 15 min to obtain a clear solution. A clean 20 ml glass vial was charged with acrylamide (1.2 g, 16.9 mmol) and 3 ml of sodium phosphate buffer (pH 7.3, 10 mM). After a brief vortex, the monomer solution was treated with AHM (0.73 g, 3.38 mmol for 20% cross-links). The mixture was sonicated for 2 min and treated with iron oxide solution (180 mg, 1 ml). The resulting monomer solution was sonicated for 5 min and added to the hexane solution under argon atmosphere with stirring. After a 20 min vigorous stirring at room temperature, the polymerization was initiated with a freshly prepared 10% ammonium per sulfate (0.065 ml) and TEMED (0.085 ml) solution. The reaction mixture was stirred at room temperature overnight.

The reaction mixture was concentrated to a thick residue under reduced pressure and the residue was diluted with 100 ml of ethanol. After a brief sonication (2 min), the mixture was transferred into an amicon stirred cell equipped with a 500 KDa polyether sulfone membrane filter and thoroughly washed with ethanol (5×180 ml). The black solid material was dried and crushed gently to a fine powder. The product was stored at 4° C. until further use. The yield of the product was 2.0 g.

### Example 13

Synthesis of Carboxylic Acid Functionalized 15% AHM Nanoparticles Encapsulating SPIO.

A clean 250 ml round bottom flask was charged with dioctylsulfosuccinate, disodium salt (3.2 g) and Brij 30 (6.4 ml) in argon purged hexanes (90 ml). The mixture was stirred at room temperature for 15 min to obtain a clear solution. A clean 20 ml glass vial was charged with acrylamide (1.2 g, 16.9 mmol), acrylic acid (80 mg, 1.1 mmol) and 3 ml of sodium phosphate buffer (pH 7.3, 10 mM). After a brief vortex, the monomer solution was treated with AHM (0.58 g, 2.7 mmol). The mixture was sonicated for 2 min and treated with iron oxide solution (1 ml, 180 mg). The resulting monomer solution was sonicated for 5 min and added to the hexane solution under argon atmosphere with stirring. After a 20 min vigorous stirring at room temperature, the polymerization was initiated with a freshly prepared 10% ammonium per sulfate (0.065 ml) and TEMED (0.085 ml) solution. The reaction mixture was stirred at room temperature overnight.

The reaction mixture was concentrated to a thick residue under reduced pressure and the residue was diluted with 100 ml of ethanol. After a brief sonication (2 min), the mixture was transferred into an amicon stirred cell equipped with a 500 KDa membrane filter and thoroughly washed with ethanol (5×180 ml). The black solid material was dried and crushed gently to a fine powder. The product was stored at 4° C. until further use. The yield of the product was 2.03 g.

### Example 14

Synthesis of Carboxylic Acid Functionalized 20% AHM Nanoparticles Encapsulating SPIO.

A clean 250 ml round bottom flask was charged with dioctylsulfosuccinate, disodium salt (3.2 g) and Brij 30 (6.4 ml) in argon purged hexanes (90 ml). The mixture was stirred at room temperature for 15 min to obtain a clear solution. A clean 20 ml glass vial was charged with acrylamide (1.2 g, 16.9 mmol), acrylic acid (80 mg, 1.1 mmol) and 3 ml of sodium phosphate buffer (pH 7.3, 10 mM). After a brief vortex, the monomer solution was treated with AHM (0.77 g, 3.6 mmol). The mixture was sonicated for 2 min and treated with iron oxide solution (1 ml, 180 mg). The resulting monomer solution was sonicated for 5 min and added to the hexane solution under argon atmosphere with stirring. After a 20 min vigorous stirring at room temperature, the polymerization was initiated with a freshly prepared 10% ammonium per sulfate (0.065 ml) and TEMED (0.085 ml) solution. The reaction mixture was stirred at room temperature overnight.

The reaction mixture was concentrated to a thick residue under reduced pressure and the residue was diluted with 100 ml of ethanol. After a brief sonication (2 min), the mixture was transferred into an amicon stirred cell equipped with a 500 KDa membrane filter and thoroughly washed with ethanol (5×180 ml). The black solid material was dried and crushed gently to a fine powder. The product was stored at 4° C. until further use. The yield of the product was 2.36 g.

### Example 15

Synthesis of Amine Functionalized 15% AHM Nanoparticles Encapsulating SPIO.

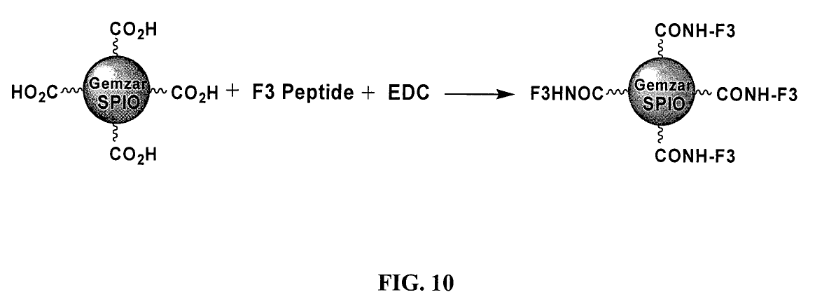

A clean 250 ml round bottom flask was charged with dioctyl sulfosuccinate (3.2 g) and Brij 30 (6.4 ml) in argon purged hexanes (90 ml). The mixture was stirred at room temperature for 20 min to obtain a clear solution. A clean 20 ml glass vial was charged with acrylamide (1.2 g, 16.9 mmol) and APMA (100 mg, 0.56 mmol) in 3 ml of sodium phosphate buffer (pH 7.3, 10 mM). After a brief vortex, the monomer solution was treated with AHM (0.56 g, 2.62 mmol for 15% cross-links). The mixture was sonicated for 2 min and the resulting monomer solution was treated with iron oxide solution (1 ml, 180 mg iron oxide) and the sonication was continued for an additional period of 5 min. The black colored solution was added to the above hexane solution under argon atmosphere. After a 20 min vigorous stirring at room temperature, the polymerization was initiated with a freshly prepared 10% ammonium per sulfate (0.065 ml) and TEMED (0.085 ml) solution. The reaction mixture was stirred at room temperature overnight. FIG. 10 illustrates the synthesis process.

The reaction mixture was concentrated to a thick residue under reduced pressure and the residue was diluted with 100 ml of ethanol. After a brief sonication (2 min), the mixture was transferred into an amicon stirred cell equipped with a 500 KDa membrane filter and thoroughly washed with ethanol (5×180 ml). The dark brown-black solid material was dried and crushed gently to a fine powder. The product was stored at 4° C. until further use. The yield of the product was 2.01 g.

### Example 16

Synthesis of Amine Functionalized 20% AHM Nanoparticles Encapsulating SPIO.

A clean 250 ml round bottom flask was charged with dioctyl sulfosuccinate (3.2 g) and Brij 30 (6.4 ml) in argon purged hexanes (90 ml). The mixture was stirred at room temperature for 20 min to obtain a clear solution. A clean 20 ml glass vial was charged with acrylamide (1.2 g, 16.9 mmol) and APMA (100 mg, 0.56 mmol) in 3 ml of sodium phosphate buffer (pH 7.3, 10 mM). After a brief vortex, the monomer solution was treated with 3-acryloyloxy-2-hydroxypropyl methacrylate (0.747 g, 3.4 mmol for 20% cross-links). The mixture was sonicated for 2 min and the resulting monomer solution was treated with iron oxide solution (1 ml, 180 mg) and the sonication was continued for an additional period of 5 min. The black colored solution was added to the above hexane solution under an argon atmosphere. After a 20 min vigorous stirring at room temperature, the polymerization was initiated with a freshly prepared 10% ammonium per sulfate (0.065 ml) and TEMED (0.085 ml) solution. The reaction mixture was stirred at room temperature overnight.

The reaction mixture was concentrated to a thick residue under reduced pressure and the residue was diluted with 100 ml of ethanol. After a brief sonication (2 min), the mixture was transferred into an amicon stirred cell equipped with a 500 KDa membrane filter and thoroughly washed with ethanol (5×180 ml). The dark brown-black solid material was dried and crushed gently to a fine powder. The product was stored at 4° C. until further use. The yield of the product was 2.5 g.

### Example 17

Synthesis of Carboxylic Acid Functionalized 15% AHM-PAA Nanoparticles Encapsulating SPIO and Gemcitabine.

A clean 250 ml round bottom flask was charged with dioctylsulfosuccinate, disodium salt (3.2 g) and Brij 30 (6.4 ml) in argon purged hexanes (90 ml). The mixture was stirred at room temperature for 15 min to obtain a clear solution. A clean 20 ml glass vial was charged with acrylamide (1.2 g, 16.9 mmol), acrylic acid (80 mg, 1.1 mmol) and 3 ml of sodium phosphate buffer (pH 7.3, 10 mM). After a brief vortex, the monomer solution was treated with AHM (0.58 g, 2.7 mmol for 15% cross-links) and solution was sonicated for 2 min. Gemcitabine (20 mg) was added to the monomer solution and mixture was gently vortexed. Iron oxide solution (1 ml, 180 mg) was added and the resulting black monomer solution was sonicated for 5 min and added to the hexane solution under an argon atmosphere with stirring. After a 20 min vigorous stirring at room temperature, the polymerization was initiated with a freshly prepared 10% ammonium per sulfate (0.065 ml) and TEMED (0.085 ml) solution. The reaction mixture was stirred at room temperature overnight.

The reaction mixture was concentrated to a thick residue under reduced pressure and the residue was diluted with 100 ml of ethanol. After a brief sonication (2 min), the mixture was transferred into an amicon stirred cell equipped with a 500 KDa membrane filter and thoroughly washed with ethanol (5×180 ml). The black solid material was dried and crushed gently to a fine powder. The product was stored at 4° C. until further use. The yield of the product was 2.4 g.

### Example 18

Synthesis of Carboxylic Acid Functionalized 20% AHM-PAA Nanoparticles Encapsulating SPIO and Gemcitabine.

A clean 250 ml round bottom flask was charged with dioctylsulfosuccinate, disodium salt (3.2 g) and Brij 30 (6.4 ml) in argon purged hexanes (90 ml). The mixture was stirred at room temperature for 15 min to obtain a clear solution. A clean 20 ml glass vial was charged with acrylamide (1.2 g, 16.9 mmol), acrylic acid (80 mg, 1.1 mmol) and 3 ml of sodium phosphate buffer (pH 7.3, 10 mM). After a brief vortex, the monomer solution was treated with AHM (0.77 g, 3.6 mmol for 20% cross-links) and solution was sonicated for 2 min. Gemcitabine (20 mg) was added to the monomer solution and the mixture was gently vortexed. Iron oxide solution (180 mg, 1 ml) was added and the resulting black monomer solution was sonicated for 5 min and added to the hexane solution under argon atmosphere with stirring. After a 20 min vigorous stirring at room temperature, the polymerization was initiated with a freshly prepared 10% ammonium per sulfate (0.065 ml) and TEMED (0.085 ml) solution. The reaction mixture was stirred at room temperature overnight.

The reaction mixture was concentrated to a thick residue under reduced pressure and the residue was diluted with 100 ml of ethanol. After a brief sonication (2 min), the mixture was transferred into an amicon stirred cell equipped with a 500 KDa membrane filter and thoroughly washed with ethanol (5×180 ml). The black solid material was dried and crushed gently to a fine powder. The product was stored at 4° C. until further use. The yield of the product was 2.52 g.

### Example 19

Synthesis of 20% AHM Cross-Linked Amine Functionalized Nanoparticles Encapsulating Gemcitabine.

A clean 250 ml round bottom flask was charged with dioctylsulfosuccinate, disodium salt (3.2 g), Brij 30 (6.4 ml) and argon purged hexanes (90 ml). The reaction mixture was stirred vigorously for 20 min under argon atmosphere. A 20 ml glass vial was charged with acrylamide (1.2 g; 16.9 mmol), 3-(aminopropyl)methacrylamide (APMA) (100 mg, 0.56 mmol) and AHM (0.747 g, 3.49 mmol for 20% cross-links) and 10 mM sodium phosphate buffer (4 ml, pH 7.3). The reaction mixture was sonicated for 5 min and gemcitabine hydrochloride (20 mg) was added to the monomer solution. The mixture was sonicated for 5 min and the clear solution was added drop-wise to the hexane solution with vigorous stirring. After a 20 min stirring at ambient temperature, the polymerization was initiated by a freshly prepared 10% ammonium per sulfate (65 μl) and TEMED (85 μl) solution. The reaction mixture was stirred at room temperature overnight.

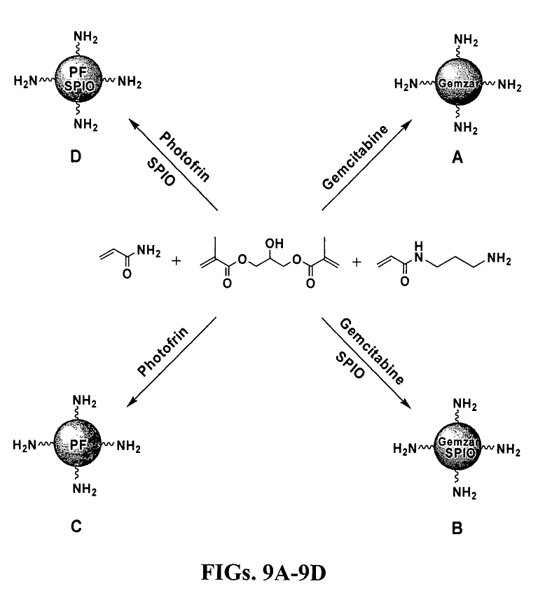

The reaction mixture was concentrated under reduced pressure to a thick semi-solid residue. The residue was treated with 50 ml of absolute ethanol and briefly sonicated to precipitate the nanoparticles. The contents of the flask were transferred into an amicon stirred cell (200 ml) equipped with a Millipore Biomax filter membrane (500,000 MWCO) and thoroughly washed with ethanol (5×50 ml). The white solid material was further dried in the amicon stirred cell and the dried nanoparticles were transferred onto a filter paper and gently crushed into a fine powder. The material was stored at 4° C. until further use. FIG. 9A illustrates the synthesis process.

### Example 20

Synthesis of 25% AHM Cross-Linked Amine Functionalized Nanoparticles Encapsulating Gemcitabine.

A clean 250 ml round bottom flask was charged with dioctylsulfosuccinate, disodium salt (3.2 g), Brij 30 (6.4 ml) and argon purged hexanes (90 ml). The reaction mixture was stirred vigorously for 20 min under argon atmosphere. A 20 ml glass vial was charged with acrylamide (1.2 g; 16.9 mmol), 3-(aminopropyl)methacrylamide (100 mg, 0.56 mmol) and AHM (934 mg, 4.36 mmol for 25% cross-links) and 10 mM sodium phosphate buffer (4 ml, pH 7.3). The reaction mixture was sonicated for 5 min and gemcitabine hydrochloride (20 mg) was added to the monomer solution. The mixture was sonicated for 5 min and the clear solution was added drop-wise to the hexane solution with vigorous stirring. After a 20 min stirring at ambient temperature, the polymerization was initiated by a freshly prepared 10% ammonium per sulfate (65 μl) and TEMED (85 μl) solution. The reaction mixture was stirred at room temperature overnight.

The reaction mixture was concentrated under reduced pressure to a thick semi-solid residue. The residue was treated with 50 ml of absolute ethanol and briefly sonicated to precipitate the nanoparticles. The contents of the flask were transferred into an amicon stirred cell (200 ml) equipped with a Millipore Biomax filter membrane (500,000 MWCO) and thoroughly washed with ethanol (5×50 ml). The white solid material was further dried in the amicon stirred cell and the dried nanoparticles were transferred onto a filter paper and gently crushed into a fine powder. The material was stored at 4° C. until further use.

### Example 21

Synthesis of Amine Functionalized 10% AHM-PAA Nanoparticles Encapsulating SPIO and Gemcitabine.

A clean 500 ml round bottom flask was charged with dioctylsulfosuccinate, disodium salt (6.4 g) and Brij 30 (12.8 ml) in argon purged hexanes (180 ml). The mixture was stirred at room temperature for 15 min to obtain a clear solution. A clean 20 ml glass vial was charged with acrylamide (2.4 g, 0.0338 mol), 3-(acryloyloxy)-2-hydroxypropyl methacrylamide (0.723 g, 0.0034 mol, 10% cross links) and 3 ml of sodium phosphate buffer (pH 7.3, 10 mM). Gemcitabine (80 mg) was added to the monomer solution and mixture was gently vortexed. Iron oxide solution (360 mg) was added and the resulting black monomer solution was sonicated for 5 min and added to the hexane solution under an argon atmosphere with stirring. After a 20 min vigorous stirring at room temperature, the polymerization was initiated with a freshly prepared 10% ammonium per sulfate (0.065 ml) and TEMED (0.085 ml) solution. The reaction mixture was stirred at room temperature overnight.

The reaction mixture was concentrated to a thick residue under reduced pressure and the residue was diluted with 100 ml of ethanol. After a brief sonication (2 min), the mixture was transferred into an amicon stirred cell equipped with a 500 KDa membrane filter and thoroughly washed with ethanol (5×180 ml). The black solid material was dried and crushed gently to a fine powder. The product was stored at 4° C. until further use. The yield of the product was 5 g.

### Example 22

Synthesis of Amine Functionalized, 10% AHM-PAA Nanoparticles Encapsulating PEG₇₅₀-PLGA, SPIO and Gemcitabine.

A clean 500 ml round bottom flask was charged with dioctylsulfosuccinate, disodium salt (6.4 g) and Brij 30 (12.8 ml) in argon purged hexanes (180 ml). The mixture was stirred at room temperature for 15 min to obtain a clear solution. A clean 20 ml glass vial was charged with acrylamide (2.4 g, 0.0338 mol), 3-(acryloyloxy)-2-hydroxypropyl methacrylamide (0.723 g, 0.0034 mol, 10% cross links) and 3 ml of sodium phosphate buffer (pH 7.3, 10 mM). Gemcitabine (80 mg) followed by PEG₇₅₀-PLGA (250 mg) were added to the monomer solution and the mixture was gently vortexed. Iron oxide solution (360 mg) was added and the resulting black monomer solution was sonicated for 5 min and added to the hexane solution under an argon atmosphere with stirring. After a 20 min vigorous stirring at room temperature, the polymerization was initiated with a freshly prepared 10% ammonium per sulfate (0.065 ml) and TEMED (0.085 ml) solution. The reaction mixture was stirred at room temperature overnight.

The reaction mixture was concentrated to a thick residue under reduced pressure and the residue was diluted with 100 ml of ethanol. After a brief sonication (2 min), the mixture was transferred into an amicon stirred cell equipped with a 500 KDa membrane filter and thoroughly washed with ethanol (5×180 ml). The black solid material was dried and crushed gently to a fine powder. The product was stored at 4° C. until further use. The yield of the product was 5 g.

### Example 23

Synthesis of Amine Functionalized 15% AHM-Nanoparticles Encapsulating SPIO and Gemcitabine.

A clean 250 ml round bottom flask was charged with dioctylsulfosuccinate, disodium salt (3.2 g) and Brij 30 (6.4 ml) in argon purged hexanes (90 ml). The mixture was stirred at room temperature for 15 min to obtain a clear solution. A clean 20 ml glass vial was charged with acrylamide (1.2 g, 16.9 mmol), APMA (100 mg, 0.56 mmol) and 3 ml of sodium phosphate buffer (pH 7.3, 10 mM). After a brief vortex, the monomer solution was treated with AHM (0.56 g, 2.6 mmol) and the solution was sonicated for 2 min. Gemcitabine (20 mg) was added to the monomer solution and mixture was gently vortexed. Iron oxide solution (1 ml, 180 mg) was added and the resulting black monomer solution was sonicated for 5 min and added to the hexane solution under argon atmosphere with stirring. After a 20 min vigorous stirring at room temperature, the polymerization was initiated with a freshly prepared 10% ammonium per sulfate (0.065 ml) and TEMED (0.085 ml) solution. The reaction mixture was stirred at room temperature overnight.

The reaction mixture was concentrated to a thick residue under reduced pressure and the residue was diluted with 100 ml of ethanol. After a brief sonication (2 min), the mixture was transferred into an amicon stirred cell equipped with a 500 KDa membrane filter and thoroughly washed with ethanol (5×180 ml). The black solid material was dried and crushed gently to a fine powder. The product was stored at 4° C. until further use. The yield of the product was 2.03 g. FIG. 9B illustrates the synthesis process.

### Example 24

Synthesis of Amine Functionalized 20% AHM-PAA Nanoparticles Encapsulating SPIO and Gemcitabine.

A clean 250 ml round bottom flask was charged with dioctylsulfosuccinate, disodium salt (3.2 g) and Brij 30 (6.4 ml) in argon purged hexanes (90 ml). The mixture was stirred at room temperature for 15 min to obtain a clear solution. A clean 20 ml glass vial was charged with acrylamide (1.2 g, 16.9 mmol), APMA (100 mg, 0.56 mmol) and 3 ml of sodium phosphate buffer (pH 7.3, 10 mM). After a brief vortex, the monomer solution was treated with 3-acryloyloxy-2-hydroxypropyl methacrylate (0.747 g, 3.6 mmol) and the solution was sonicated for 2 min. Gemcitabine (20 mg) was added to the monomer solution and the mixture was gently vortexed. Iron oxide solution (1 ml, 180 mg) was added and the resulting black monomer solution was sonicated for 5 min and added to the hexane solution under argon atmosphere with stirring. After a 20 min vigorous stirring at room temperature, the polymerization was initiated with a freshly prepared 10% ammonium per sulfate (0.065 ml) and TEMED (0.085 ml) solution. The reaction mixture was stirred at room temperature overnight.

The reaction mixture was concentrated to a thick residue under reduced pressure and the residue was diluted with 100 ml of ethanol. After a brief sonication (2 min), the mixture was transferred into an amicon stirred cell equipped with a 500 KDa membrane filter and thoroughly washed with ethanol (5×180 ml). The black solid material was dried and crushed gently to a fine powder. The product was stored at 4° C. until further use. The yield of the product was 2.53 g.

### Example 25

Synthesis of Amine Functionalized 20% AHM Nanoparticles Encapsulating Photofrin.

A clean 250 ml round bottom flask was charged with dioctyl sulfosuccinate, sodium salt (3.2 g), Brij 30 (6.4 ml) and argon purged hexanes (90 ml). The reaction mixture was stirred under argon atmosphere for 20 min. A clean 20 ml glass vial was charged with acrylamide (1.2 g, 0.0169 mol), AHM (0.72 g, 0.00338 mol, 20% cross-links) and 10 mM sodium phosphate buffer (3.5 ml, pH 7.3). After a brief sonication, 3-(aminopropyl)methacrylamide (100 mg, 0.559 mmol, FW 178.7) was added to the acrylamide solution and sonicated for 5 min. Photofrin (10 mg) was added to the clear monomer solution and the resulting deep brown-red solution was briefly sonicated. The monomer solution was added to the hexane reaction mixture and the solution was stirred under argon for 20 min. The polymerization was initiated with a freshly prepared 10% ammonium per sulfate (65 μL) and TEMED (85 μL) solution. The reaction mixture was gently stirred under argon atmosphere overnight.

The reaction mixture was concentrated under reduced pressure and the residue was treated with 100 ml of ethanol. The precipitated nanoparticle mixture was transferred into an Amicon stirred cell (200 ml, equipped with a Biomax 500,000 MWCO membrane) and thoroughly washed with ethanol (5×180 ml). After the fifth ethanol wash, the solid material in the stirred cell was allowed to dry under nitrogen pressure (10 psi) overnight. The dried product was transferred onto a weighing paper and gently crushed into a fine powder. The yield of the product was 1.4 g. FIG. 9C illustrates the synthesis process.

### Example 26

Synthesis of Amine Functionalized 20% AHM Nanoparticles Encapsulating Photofrin and SPIO.

A clean 250 ml round bottom flask was charged with dioctyl sulfosuccinate, sodium salt (3.2 g), Brij 30 (6.4 ml) and argon purged hexanes (90 ml). The reaction mixture was stirred under argon atmosphere for 20 min. A clean 20 ml glass vial was charged with acrylamide (1.2 g, 0.0169 mol), 3-(acryloyloxy)-2-hydroxypropyl methacrylamide (0.72 g, 0.00338 mol, 20% cross links) and 10 mM sodium phosphate buffer (3.5 ml, pH 7.3). After a brief sonication, 3-(aminopropyl)methacrylamide (100 mg, 0.559 mmol, FW 178.7) was added to the acrylamide solution and sonicated for 5 min. Photofrin (10 mg) was added to the clear monomer solution and the resulting deep brown-red solution was briefly sonicated. SPIO (90 mg, 0.5 ml) was added to the deep brown-red monomer solution and the resultant black solution was sonicated for 2 min. The monomer mixture was added to the hexane reaction mixture and the solution was stirred under argon for 20 min. The polymerization was initiated with a freshly prepared 10% ammonium per sulfate (65 μL) and TEMED (85 μL) solution. The reaction mixture was gently stirred under argon atmosphere overnight.

The reaction mixture was concentrated under reduced pressure and the residue was treated with 100 ml of ethanol. The precipitated nanoparticle solution was transferred into an Amicon stirred cell (200 ml, equipped with a Biomax 500,000 MWCO membrane) and thoroughly washed with ethanol (5×180 ml). After the fifth ethanol wash, the solid material in the stirred cell was allowed to dry under nitrogen pressure (10 psi) overnight. The dried product was transferred onto a weighing paper and gently crushed into a fine powder. The yield of the product was 2.35 g. FIG. 9D illustrates the synthesis process.

### Example 27

F3 (KDEPQRRSARLSAKPAPPKPEPKPKKAPAKK (SEQ ID NO:1)) Targeted Nanoparticles Encapsulating SPIO and Gemcitabine

A clean glass vial (20 ml) was charged with 300 mg of SPIO and gemcitabine encapsulated carboxy functionalized nanoparticles in 15 ml of PBS. The solution was treated with N-Ethyl-N′-(3-dimethylaminopropyl)-carbodimide (4.4 mg, EDC) in small portions over a period of 30 min. The reaction mixture was stirred at room temperature for and additional 30 min and then F3 peptide (20 mg, 0.0058 mmol) was added in one portion with stirring. The resulting reaction mixture was stirred at room temperature overnight. The reaction mixture was transferred into an amicon stirred cell and thoroughly washed with PBS (10×45 ml). The concentrated sample was filtered through a 0.2μ filter and used for in vivo and in vitro experiments. FIG. 10 illustrates the synthesis process.

### Example 28

F3 Peptide Conjugated Nanoparticles Encapsulating SPIO and Gemcitabine.

The amine-functionalized 20% AHM cross-linked nanoparticles encapsulating gemcitabine and SPIO (500 mg) were suspended in PBS and sonicated for 20 min. The solution was transferred into an amicon stirred cell (200 ml) equipped with a 500,000 MWCO biomax filter membrane and thoroughly washed with PBS. The concentrated nanoparticle solution (40 mg/ml) was transferred into a clean round bottom flask (100 ml). The solution was treated with sulfo SMCC (19.2 mg, 0.044 mmol) and the reaction mixture was stirred at room temperature for 1 h.

A clean 20 ml glass vial was charged with F3 peptide (KDEPQRRSARLSAKPAPPKPEPKPKKAPAKK (SEQ ID NO:1)) (25 mg=0.0075 mmol, FW 3433) in PBS (10 ml). The solution was treated with 2-iminothiolane (1 mg=0.0075 mmol, FW 137.6) and the reaction mixture was gently agitated for 30 min at room temperature. The reaction mixture was carefully added to the above nanoparticle solution and the solution was gently agitated overnight.

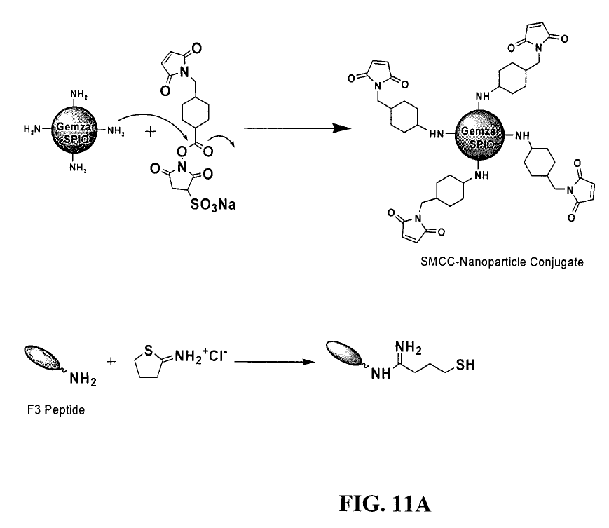

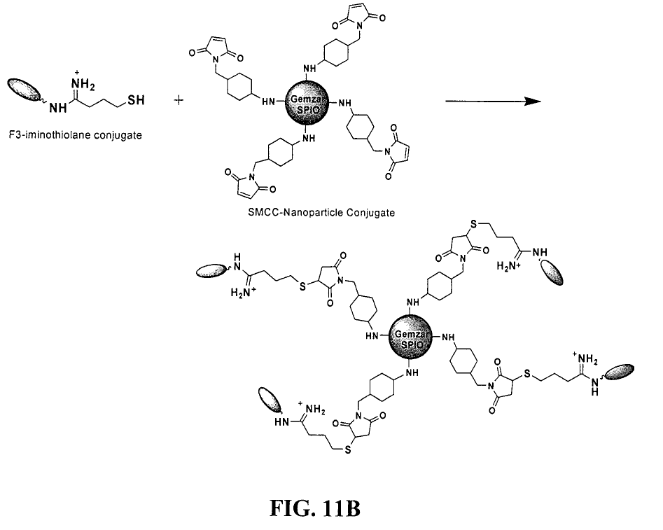

The reaction mixture was treated with L-cysteine (5 mg, 0.04 mmol) and stirred at room temperature for 30 min. The reaction mixture was transferred into an amicon stirred cell (50 ml) equipped with a biomax filter membrane and thoroughly washed with PBS. The concentrated sample (71 mg/ml) was filtered through a 0.2μ filter and the material was stored at 4° C. FIGS. 11a-11b illustrate the synthesis procedure.

### Example 29

Synthesis of F3 Targeted 10% AHM Functionalized Nanoparticles Encapsulating Photofrin and SPIO.

A clean 20 ml glass vial was charged with amine functionalized nanoparticles encapsulating Photofrin and SPIO (800 mg, 0.32 mmol NH₂ groups) in PBS. The reaction mixture was treated with 28 mg of SMCC (0.064 mmol) and the mixture was gently stirred at room temperature for 2 h. The reaction mixture was carefully transferred into an amicon stirred cell (50 ml) equipped with a 500,000 MWCO biomax filter membrane and concentrated to a small volume (˜5 ml). The concentrated solution was diluted with PBS (50 ml) and concentrated to a small volume. The washings of the SMCC conjugate were repeated for 4 more times to remove any unreacted material from the reaction mixture. The concentrated solution (10 ml) was directly used in the next step.

A clean 20 ml glass vial was charged with F3 peptide (25 mg=0.0075 mmol) in PBS (10 ml). The solution was treated with 2-iminothiolane (1 mg-0.0075 mmol) and the reaction mixture was gently agitated for 30 min at room temperature. The peptide mixture was carefully added to the above nanoparticle solution and the solution was gently agitated for overnight.

The peptide conjugate was transferred into amicon stirred cell and thoroughly washed with PBS (4×100 ml). The concentrated solution (88 mg/ml) was passed through 0.2 micron filters and stored at 4° C. until further use.

### Example 30

Synthesis of F3-C Peptide Conjugated Nanoparticles Encapsulating Photofrin.

In a clean 20 ml glass vial amine-functionalized 20% AHM cross-linked nanoparticles encapsulating Photofrin and SPIO (200 mg) were suspended in PBS. The mixture was sonicated for 20 min and the solution was transferred into an amicon stirred cell (200 ml) equipped with a 500,000 MWCO biomax filter membrane and thoroughly washed with PBS. The concentrated nanoparticle solution (23.8 mg/ml) was transferred into a clean glass vial (20 ml). The solution was treated with sulfo SMCC (23 mg, 0.053 mmol) and the reaction mixture was stirred at room temperature for 1 h.

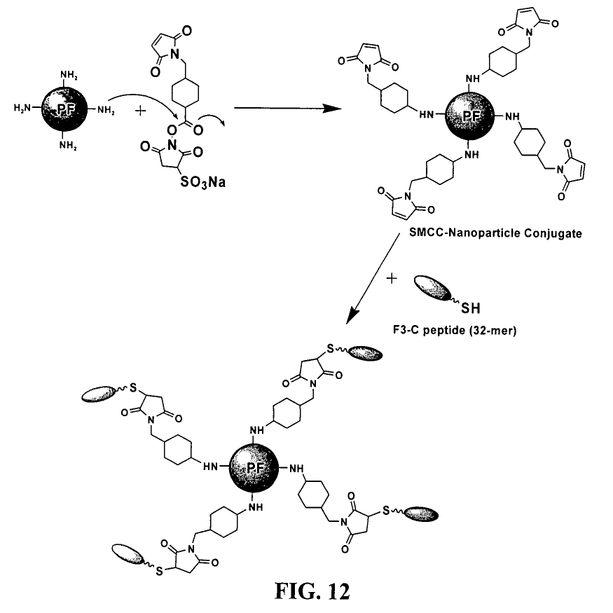

A clean 20 ml glass vial was charged with F3-C peptide (KDEPQRRSARLSAKPAPPKPEPKPKKAPAKKC (SEQ ID NO: 2)) (12 mg=0.003 mmol, FW 3566) in 0.1M sodium phosphate buffer containing 0.15 M sodium chloride solution (5 ml). The solution was carefully added to the above nanoparticle solution and the solution was gently agitated for 2 h. The reaction mixture was treated with L-cysteine (5 mg, 0.04 mmol) and stirred at room temperature for 30 min. The reaction mixture was transferred into an amicon stirred cell (50 ml) equipped with a biomax filter membrane and thoroughly washed with PBS. The concentrated sample (33 mg/ml) was filtered through 0.2μ filters and the material was stored at 4° C. FIG. 12 illustrates the synthesis procedure.

### Example 31

Synthesis of C-F3 Targeted Nanoparticles Encapsulating Gemcitabine and SPIO.

A clean 100 ml RB flask was charged with amine functionalized nanoparticles encapsulating gemcitabine and SPIO (450 mg, 0.23 mmol NH₂ groups) in PBS. The reaction mixture was treated with 26 mg of SMCC (0.059 mmol) and the mixture was gently stirred at room temperature for 2 h. The reaction mixture was carefully transferred into an amicon stirred cell (50 ml) equipped with a 500,000 MWCO biomax filter membrane and concentrated to a small volume (˜5 ml). The concentrated solution was diluted with PBS (50 ml) and concentrated to a small volume. The washings of the SMCC conjugate were repeated for 2 more times to remove any unreacted material from the reaction mixture. The concentrated solution (10 ml) was placed in a 20 ml glass vial and treated with CKDEPQRRSARLSAKPAPPKPEPKPKKAPAKK (SEQ ID NO:3) (cysteine terminal F3 peptide, C-F3, 45 mg, 0.0127 mmol). The reaction mixture was stirred at 4° C. overnight.

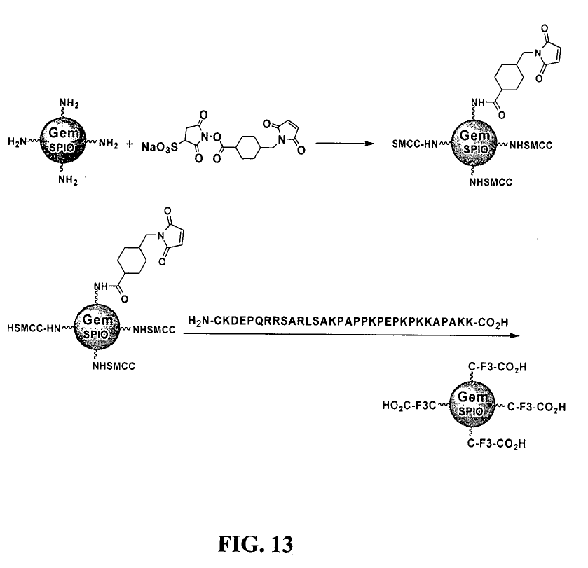

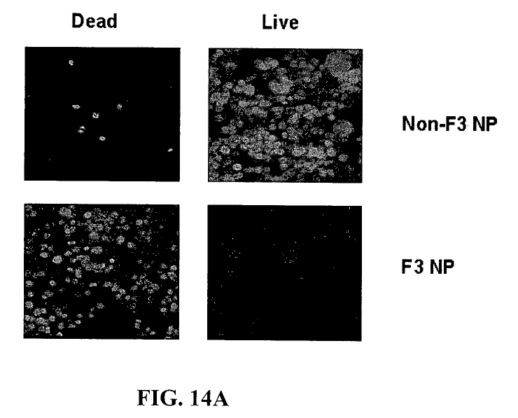

The peptide conjugate was transferred into amicon stirred cell and thoroughly washed with PBS (4×100 ml). The concentrated solution (6.5 ml, 69 mg/ml) was passed through 0.2μ filters and stored at 4° C. until further use. The peptide conjugate was used for radiosensitization experiments. FIG. 13 illustrates the synthesis procedure.

### Example 32

Synthesis of DTPA Conjugated Nanoparticles

A clean round bottom flask (50 ml) was charged with diethylenetriamine penta acetic acid (DTPA) (3.2 g, 8.05 mmol) in 15 ml of acetonitrile. The suspension was treated with triethylamine (5.6 ml, 40 mmol) and the mixture was stirred at 60° C. for 30 min to obtain a clear solution. The solution was treated with isobutyl chloroformate (1.05 g, 8 mmol, −30° C.) and after 30 min the mixture was added to a solution of amine functionalized nanoparticles (2 g, 2.68 mmol amine groups) in 0.1 M sodium bicarbonate solution (50 ml). The reaction mixture was stirred at room temperature for 12 h. The solution was concentrated to about half of its original volume and subjected to dialysis against 0.05 M oxalic acid (pH 2) for 24 h (3 exchanges) and then against 0.03 M sodium bicarbonate (pH 8) for 24 h (3 exchanges). After the dialysis, the nanoparticle solution was transferred into an amicon stirred cell (200 ml size equipped with a 500,000 MWCO polyether sulfone membrane) and thoroughly washed (6×180 ml). The nanoparticle solution was used directly in Example 33.

### Example 33

Synthesis of Gadolinium-DTPA Conjugated Nanoparticles

The DTPA conjugated nanoparticle solution (2 g, 2.68 mmol DTPA groups) was placed in a round bottom flask (250 ml) and GdCl₃.6H₂O (996 mg, 2.68 mmol) was added drop-wise in 15 ml of water. After the addition, the pH of the solution was adjusted to 7.0. The reaction mixture was stirred at room temperature for 5 h and at 60° C. for 1 h. The reaction mixture was transferred into an amicon stirred cell (200 ml, equipped with a 500,000 MWCO polyether sulfone membrane) and concentrated to 20 ml. The material was diluted with water (160 ml) and filtered. After each wash an aliquot of the filtrate was checked with Arsenazo reagent to see any free gadolinium content. The concentrated sample (15 ml) was passed through 0.8 and 0.45μ filters and the relaxivity was measured by MRI.

### Example 34

Synthesis of Nanoparticles Encapsulating Gadomer-17 (0.25 mmol Gd Content).

A clean 250 ml round bottom flask was charged with dioctyl sulfosuccinate (3.2 g), Brij 30 (6.4 ml) and argon purged hexanes (90 ml). The mixture was stirred under argon for 20 min. A 20 ml glass vial was charged with acrylamide (1.2 g, 16.88 mmol) and sodium phosphate buffer (3.5 ml, 10 mM, pH 7.3). The mixture was sonicated for 2 min to obtain a clear solution. Gadomer-17 (0.5 ml, 0.25 mmol Gd) was added to the monomer solution followed by AHM (0.9 g, 4.2 mmol) and the mixture was sonicated for 2 min to get a slightly turbid solution. Methanol (0.5 ml) was added to the monomer solution and sonicated for 2 min to obtain a clear solution. This monomer solution was added to the hexane solution and the resultant mixture was stirred vigorously for 20 min under argon atmosphere. The clear solution was treated with a freshly prepared 10% ammonium per sulfate (65 μL) and TEMED (85 μL) solution to initiate the polymerization. The reaction mixture was stirred for 12 h and concentrated under reduced pressure to give a thick residue. This was taken up in ethanol (160 ml) and the separated solid was thoroughly washed with ethanol (5×180 ml) in an amicon stirred cell. The material was dried and crushed into a fine powder. The yield of the product was 2.07 g (87%). The material was stored at 4° C. until further use.

### Example 35

Synthesis of Nanoparticles Encapsulating Gadomer-17 (0.5 mmol Gd Content).

A clean 250 ml round bottom flask was charged with dioctyl sulfosuccinate (3.2 g), Brij 30 (6.4 ml) and argon purged hexanes (90 ml). The mixture was stirred under argon for 20 min. A 20 ml glass vial was charged with acrylamide (1.2 g, 16.88 mmol) and sodium phosphate buffer (3.5 ml, 10 mM, pH 7.3). The mixture was sonicated for 2 min to obtain a clear solution. Gadomer-17 (1 ml, 0.5 mmol Gd) was added to the monomer solution followed by AHM (0.9 g, 4.2 mmol) and the mixture was sonicated for 2 min to get a slightly turbid solution. Methanol (0.5 ml) was added to the monomer solution and sonicated for 2 min to obtain a clear solution. This monomer solution was added to the hexane solution and the resultant mixture was stirred vigorously for 20 min under argon atmosphere. The clear solution was treated with a freshly prepared 10% ammonium per sulfate (65 μL) and TEMED (85 μL) solution to initiate the polymerization. The reaction mixture was stirred for 12 h and concentrated under reduced pressure to give a thick residue. This was taken up in ethanol (160 ml) and the separated solid was thoroughly washed with ethanol (5×180 ml) in an amicon stirred cell. The material was dried and crushed into a fine powder. The yield of the product was 2.26 g (87%). The material was stored at 4° C. until further use.

### Example 36

Cleanup of AHM Nanoparticles Encapsulating Gadomer-17.

400 mg of each sample was suspended in 50 ml of water and sonicated for 15 min. The solution was passed through 0.45 and 0.2μ filters and transferred into an amicon stirred cell (200 ml, 500,000 MWCO polyether sulfone membranes). The solution was washed repeatedly until no free gadomer-17 was observed in the filtrate. The solution was concentrated to a small volume (6 ml) and again filtered through 0.2μ syringe filter. Appropriate dilutions were made for MRI testing.

### Example 37

Synthesis of 25% Cross Linked Carboxylic Acid Functionalized AHM Nanoparticles

A clean 500 ml round bottom flask was charged with AOT (6.4 g) and Brij 30 (12.8 ml) in argon purged hexanes (180 ml). The mixture was stirred at room temperature for 15 min to obtain a clear solution. A clean 20 ml glass vial was charged with acrylic acid (1.2 g, 16.7 mmol) and acrylamide (1.2 g, 16.9 mmol) in 8 ml of sodium phosphate buffer (pH 7.3, 10 mM). After a brief vortex, the monomer solution was treated with AHM (1.8 g, 8.4 mmol). The mixture was sonicated for 2 min and the resulting monomer solution was added to the above hexane solution under argon atmosphere with stirring. After a 20 min vigorous stirring at room temperature, the polymerization was initiated with a freshly prepared 10% ammonium per sulfate (0.065 ml) and TEMED (0.085 ml) solution. The reaction mixture was stirred at room temperature for 12 h.

The reaction mixture was concentrated to a thick residue under reduced pressure and the residue was diluted with 100 ml of ethanol. After a brief sonication (2 min), the precipitated particles were transferred into an amicon stirred cell equipped with 500 KDa membrane filter and thoroughly washed with ethanol (5×180 ml). The material was dried and crushed gently to a fine powder to give 4.46 g (105%) of white solid material.

### Example 38

Conversion of Acid Functionalized Nanoparticles into Triamine Functionalized Nanoparticles

The nanoparticle powder from Example 37 (1 g, 3.74 mmol carboxylic acid groups) was suspended in 50 ml of water and sonicated for 15 min. The clear slight milky solution was filtered through 0.2μ filters and placed in a 100 ml round bottom flask. N-hydroxy succinimide (0.43 g, 3.74 mmol), EDC (0.717 g, 3.74 mmol) and DMAP (0.1 g) were added to the nanoparticle solution and stirred at 4° C. for 30 min. Triethylene tetramine hydrate (1.83 g, 11.2 mmol, excess) was added to the reaction mixture and the mixture was stirred at room temperature for 12 h.

The reaction mixture was transferred into an amicon stirred cell (200 ml) equipped with a 500,000 MWCO PES membrane and extensively washed with water. When the filtrate was negative to amine test, the solution was concentrated to a small volume (40 ml) and stored at 4° C.

### Example 39

Reaction of Triamine Functionalized Nanoparticles with DTPA Dianhydride

The triamine functionalized nanoparticle solution from Example 38 (20 ml, 0.5 g) was placed in a 100 ml round bottom flask and treated with 2.1 ml of triethylamine with stirring. The nanoparticle solution was added drop wise to a suspension of DTPA dianhydride (5 g, 14 mmol) in water (20 ml). A clear solution was obtained after complete addition of the nanoparticle solution. The reaction mixture was stirred at room temperature for overnight. The reaction mixture was repeatedly washed with water (6×180 ml) in an amicon stirred cell equipped with a 500,000 MWCO PES membrane to remove excess DTPA and the concentrated solution (20 ml) was directly used Example 40.

### Example 40

Synthesis of Gadolinium DTPA Nanoparticles

The nanoparticle solution obtained in the above step was placed in a 100 ml round bottom flask and treated with gadolinium chloride hexahydrate (695 mg, 1.87 mmol) in water (15 ml). The reaction mixture was stirred at room temperature for 2 h and an additional 695 mg of gadolinium chloride hexahydrate in 15 ml of water was added. The pH of the reaction was adjusted to 7 and the mixture was stirred at room temperature overnight. The reaction mixture was filtered through 0.2μ filters and extensively washed with water to remove any excess gadolinium chloride. Each filtrate was tested for any free Gadolinium by arsenazo reagent, and when the test was negative, the nanoparticle solution was concentrated to a small volume (6 ml). The solution was filtered through a 0.2μ filter and stored at 4° C. until further use.

### Example 41

Chemically Induced Degradation of Glycerol(bis) Acrylate Nanoparticles.

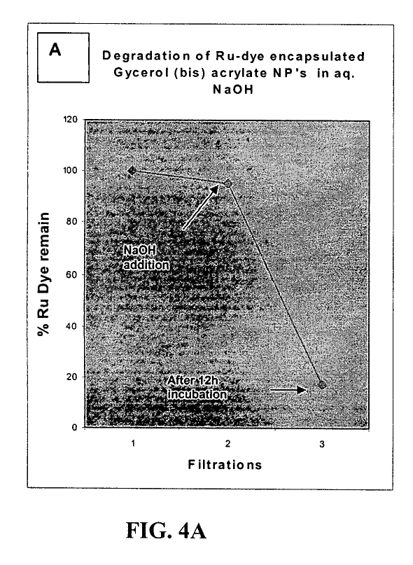

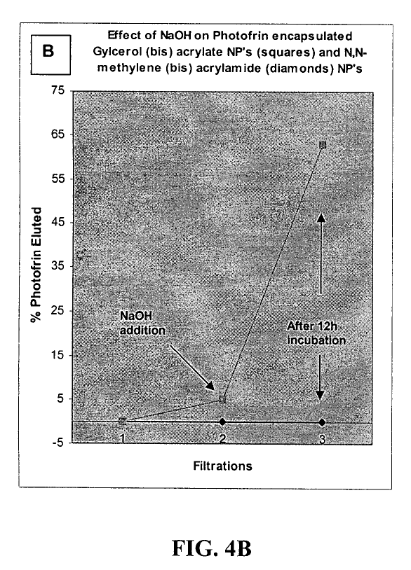

100 mg of Ru-loaded glycerol(bis)acrylate (GBA) nanoparticles were suspended in 10 ml PBS and filtered. The residue was treated with 10 mL of 1 M sodium hydroxide incubated for 12 h intervals and filtered. FIG. 4A shows the percentage of Ru dye remaining in the GBA nanoparticles following treatment with NaOH and filtration at 12 hour intervals.

100 mg of Photofrin GBA nanoparticles were suspended in 10 ml PBS and filtered. The residue was treated with 10 mL of 1 M sodium hydroxide and incubated for 12 h intervals and filtered. FIG. 4B shows the percentage of Photofrin eluted at 12 hour intervals for GBA nanoparticles (squares) and nanoparticles made with a non-biodegradable cross-linker (N,N-methylene (bis)acrylamide) and acrylamide as the polymer backbone (diamonds).

### Example 42

Synthesis of Nanoparticles Encapsulating Photofrin (or Ruthenium Dye).

The following synthesis is on a 2 g scale and it can be extended from multi gram to kilogram level.

A clean 20 ml glass vial was charged with acrylamide (1.2 g) and 4 ml of sodium phosphate buffer (10 mM, pH 7.3). The suspension was sonicated for 2 min to obtain a clear solution. Glycerol(bis)acrylate (0.53 g) was added to the reaction mixture and sonicated for an additional 5 min. Photofrin (or Ruthenium dye (Ru)) was added and the mixture was sonicated for an additional 5 min. The resulting slightly turbid monomer solution was added to a 250 ml round bottom flask containing an argon-purged, well stirred solution of dioctyl sulfosuccinate (3.2 g) and Brij 30 (6.4 ml) in hexanes (90 ml). After a 10 min stirring under an argon blanket at room temperature, the reaction mixture was treated with freshly prepared aqueous ammonium per sulfate (65 μl, 10%) and N,N,N,N-tetra methyl-ethylenediamine (TEMED) (85 μl) to initiate the polymerization. The reaction mixture was gently stirred at room temperature overnight to ensure complete polymerization.

The reaction mixture was concentrated to a thick residue and re-suspended in ethanol (100 ml). The precipitated particles were filtered and thoroughly washed with ethanol (5×160 ml) in an Amicon stirred cell equipped with a Biomax filter membrane (500 Kda, filtration pressure 10 psi, nitrogen). The solid material was transferred onto a Whatman filter paper, gently crushed into a fine powder and subjected to air-drying until a constant weight was observed (3-4 hrs). The product (dark brown free flowing powder in the case of photofrin and light pink powder for Ru dye) can be stored at 4° C. for extended periods of time.

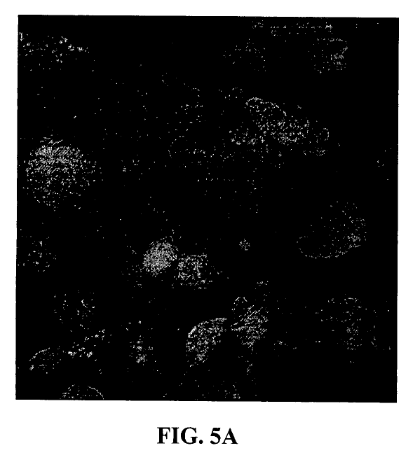

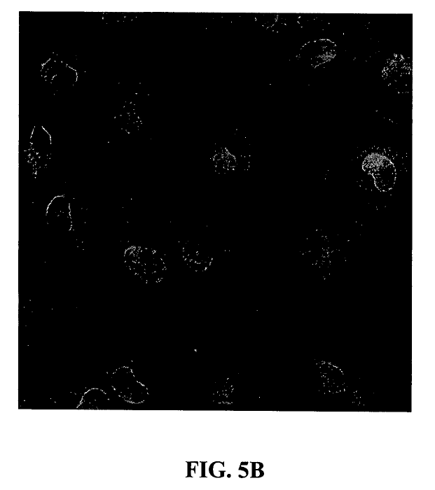

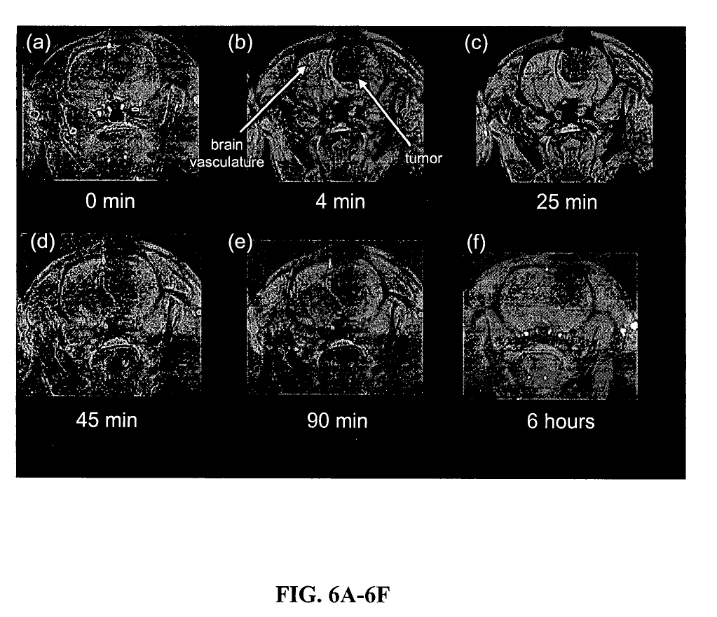

The product was suspended in water (20 mg/ml) and sonicated to get a homogenous solution. The solution was transferred into an amicon stirred cell equipped with a Biomax (500 Kda) filter membrane and thoroughly washed with water (5×150 ml). The concentrated sample (˜50 mg/ml) was passed through 0.45μ and 0.2μ filters and stored at 4° C. until further use. FIGS. 5A and 5B show the effect of Ruthenium dye (Ru) encapsulated in glycerol(bis) acrylate nanoparticles in laser assisted cell kill of Rat 9L glioma cells. 5A shows the effects of blank glycerol(bis)acrylate nanoparticles and laser light. 5B shows the effects of Ru dye encapsulated in glycerol(bis)acrylate nanoparticles and laser light.

### Example 43

Synthesis of Poly(Vinyl Pyrrolidone)-Citrate Nanoparticles

Synthesis of 1,5-Divinyl Citrate

In a 250 mL round bottom flask, citric acid (19.2 g, 100 mmol) was dissolved in anhydrous dimethyl sulfoxide (50 mL) and added vinyl acetate (23.0 mL, 250 mmol) with vigorous stirring. Concentrated sulfuric acid (0.5 mL) was added drop-wise at room temperature under argon. The reaction mixture was stirred at room temperature for 48 h under argon.

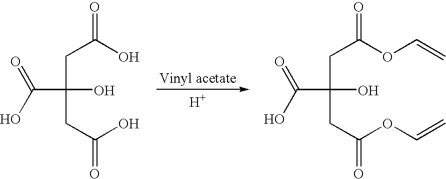

All volatiles were evaporated and lime water (30.0 g of calcium carbonate suspended in 400 mL of water) was used to neutralize the solution to pH 7.0. A white precipitate was formed which was filtered off and discarded. The filtrate was concentrated under vacuum to give a residue which was diluted with water (40 mL). The mixture was sonicated and filtered. The filtered solid was discarded and filtrate was kept. Concentrated hydrochloric acid was added to bring to pH 0. A white precipitate was formed which was filtered and kept. The precipitate was dissolved in saturated sodium bicarbonate solution (10 mL). The solution was washed three times with chloroform (3×50 mL). Then, concentrated hydrochloric acid was added again to bring the solution to pH 0. A sticky white precipitate was formed which was dried under vacuum to give 1,5-divinyl citrate as sticky solid (6.38 g, 24%).

### Example 44

Blank Citrate Cross Linked PVP Nanoparticles

An oven dried 250 mL round bottom flask was charged with hexane (100 mL) and was stirred under a constant purge of argon. Aerosol OT (3.40 g) and Brij 30 (6.5 mL) was added to the reaction flask and stirring was continued under argon until a uniform solution was formed. A 20 mL glass sample tube was charged with vinylpyrrolidinone (2.20 g; 20 mmol) and was dissolved in 0.1 M sodium phosphate buffer (3.5 mL, pH 7.4) by sonication. Divinyl citrate (1.064 g; 4 mmol; 20 mole % with respect to vinylpyrrolidinone) was added to the vinylpyrrolidinone solution and sonication was continued until a uniform solution resulted. Azobis(2-methylpropionamidine)dihydrochloride (ABMP.HCl 54.0 mg, 0.02 mmol) was added to the mixture as polymerization initiator and sonicated to get a clear solution. The uniform suspension was added to the hexane reaction mixture and was stirred vigorously for 15 minutes at room temperature under argon. The reaction mixture was stirred vigorously at room temperature for 10 min and at 50° C. for 12 h under argon. The solution was then cooled to room temperature and hexane was removed under reduced pressure to give a thick syrupy residue which was diluted with ethanol (100 mL). The mixture was sonicated and the separated particles were washed in an Amicon stirred cell (500 K cut off filter, Millipore, 200 mL) with ethanol (5×150 mL). The white nanoparticles obtained were dried under nitrogen and gently crushed to a fine powder (2.4 g). The material was stored at 4° C.

### Example 45

Citrate Cross Linked PVP Nanoparticles Encapsulating Ru-Dye

An oven dried 250 mL round bottom flask was charged with hexane (100 mL) and was stirred under a constant purge of argon. Aerosol OT (3.40 g) and Brij 30 (6.5 mL) was added to the reaction flask and stirring was continued under argon until a uniform solution was formed. A 20 mL glass sample tube was charged with vinylpyrrolidinone (2.20 g; 20 mmol) and was dissolved in 0.1 M sodium phosphate buffer (3.5 mL, pH 7.4) by sonication. Divinyl citrate (1.064 g; 4 mmol; 20 mole % with respect to monomer) was added to the vinylpyrrolidinone solution and sonication was continued until a uniform solution resulted. Azobis(2-methylpropionamidine)dihydrochloride (ABMP.HCl 54.0 mg, 0.02 mmol) was added to the mixture as polymerization initiator followed by Ru-dye (20 mg) and sonicated to get a clear dark red solution. The uniform suspension was added to the hexane reaction mixture and was stirred vigorously for 15 minutes at room temperature under argon. The reaction mixture was stirred vigorously at room temperature for 10 min and at 50° C. for 12 h under argon. The solution was then cooled to room temperature and hexane was removed under reduced pressure to give a thick syrupy residue which was diluted with ethanol (100 mL). The mixture was sonicated and the separated particles were washed in an Amicon stirred cell (500K cut off filter, Millipore, 200 mL) with ethanol (5×150 mL). The white nanoparticles obtained were dried under nitrogen and gently crushed to a fine powder (2.6 g). The material was stored at 4° C.

### Example 46

Degradation Citrate Cross Linked PVP Nanoparticles Encapsulated Ru-Dye with NaOH.

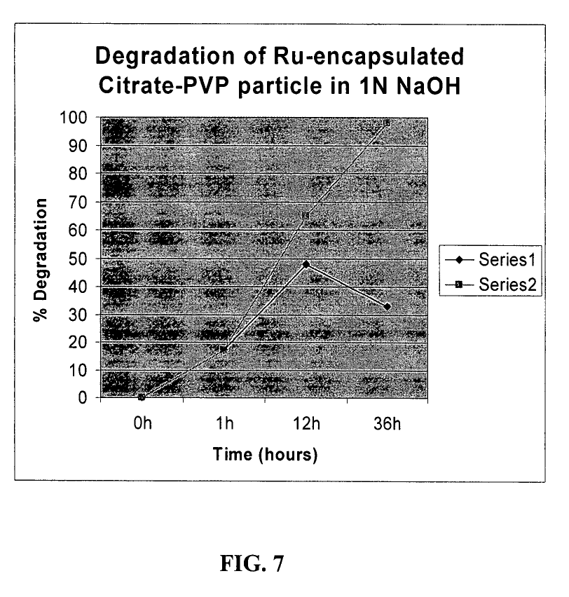

Ru-dye containing nanoparticles (200 mg) were dissolved in 1 N sodium hydroxide solution and filtered through 100 K cutoff membrane and the filtrate and mother solution were monitored at 1 h, 12 h and 36 h by UV spectroscopy. The amount of dye coming out in the filtrate indicated the percentage of degradation over time. FIG. 7 shows the percent degradation over the time period 0-36 hours. Series 1 shows the amount of dye released in each measurement and series 2 indicates the total amount of dye released over time.

### Example 47

In Vitro Studies Demonstrating Targeting Dependent Killing of Tumor Cells Using Photodynamic Therapy (PDT)

To check the nanoparticle preparations to ensure that all the expected characteristics were present in each preparation, targeting capacity and therapeutic efficacy were determined in tissue culture cells. A monolayer of MDA-435 cells was first generated. F3 Targeted and non-targeted nanoparticles containing photofrin were then added, and after a given incubation period (4 hours or 5 seconds), cells are washed to remove unbound nanoparticles, and then exposed laser light for a given period of time to activate the photofrin.

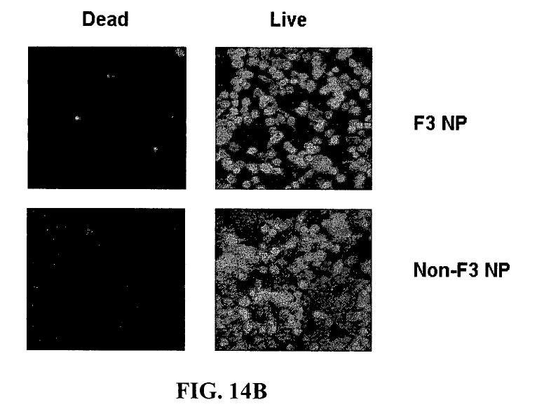

Data shown in FIG. 14a demonstrate that F3 targeted nanoparticles (bottom two panels) killed a large percentage of the tumor cells, but non-targeted nanoparticles (top two panels) were much less effective. FIG. 14a represents fluorescent photographs using a live/dead fluorescent stain. Non F3-targeted particles (top two panels) show very few dead cells, and far more live cells, compared to the F3-targeted nanoparticles. From these results, it appears that targeted is required to efficiently kill MDA-435 cells.

To demonstrate that the killing observed in the above experiment was due to binding of nanoparticles to cells, the experiment was repeated, except that the nanoparticles were washed off after a 5 second incubation, thus not providing enough time for the targeted cells to bind. The results presented in FIG. 14b demonstrate almost no cell kill, even with the targeted nanoparticles. This demonstrates that the killing observed with the targeted nanoparticles above was due to binding of the particles (and not some non-specific toxicity) to the cells since removal of the particles prior to complete binding negated all killing activity.

These in vitro studies demonstrate that the nanoparticle preparations had the desired characteristics, i.e. that they, in an F3 dependent manner, bound to cancer cells, and that the encapsulated photofrin resulted in a laser and dose dependent killing of cancer cells.

### Example 48

In Vivo Therapy of Glioma Using Photofrin-Containing Nanoparticles.

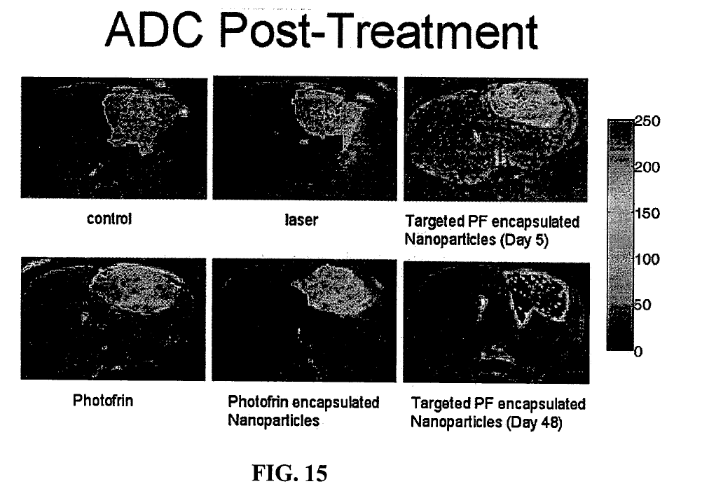

Rats harboring intracerebral 9L gliomas, were allowed to grow to approximately 50 microliters in size, as quantitated using multi-slice T2-weighted MRI. Rats were divided into the following groups: sham (untreated); laser exposure only; intravenous administered photofrin (no nanoparticles); photofrin-encapsulated nanoparticles; and photofrin-encapsulated nanoparticles with F3 targeting (n=5/group). Diffusion MRI scans were obtained prior to treatment and every 2-3 days following treatment. Treatment with laser administration consisted of 750 mW of laser light administered at 630 nm for 7.5 minutes of total exposure time. This was accomplished through the same 1 mm diameter burr hole which was initially made to inject the 9L tumor cells to initiate tumor growth. The depth of the cylindrical laser tip was adjusted, based upon the MRI scan data, to be at the base of the tumor mass in order to optimize light delivery to the overall tumor mass. Diffusion MRI scans were acquired in order to assess in real time, the effectiveness of treatment. Increased diffusion values within the tumor mass reflect areas of cell kill (necrosis) and the magnitude of the increase is proportional to the level of cell killing. As shown in the MRI scans in FIG. 15, control diffusion tumor values are on average at approximately 100 apparent diffusion coefficient units (ADC). FIG. 15 shows ADC overlay maps of control, laser, Photofrin, Photofrin/nanoparticles, and Photofrin/F3-nanoparticle treated 9L tumors, all at 5 days post laser treatment. Also shown is a Photofrin/F3-nanoparticle treated tumor at 48 days post treatment revealing that complete killing of the tumor mass had occurred, resulting in a cystic region where the tumor mass had been located.

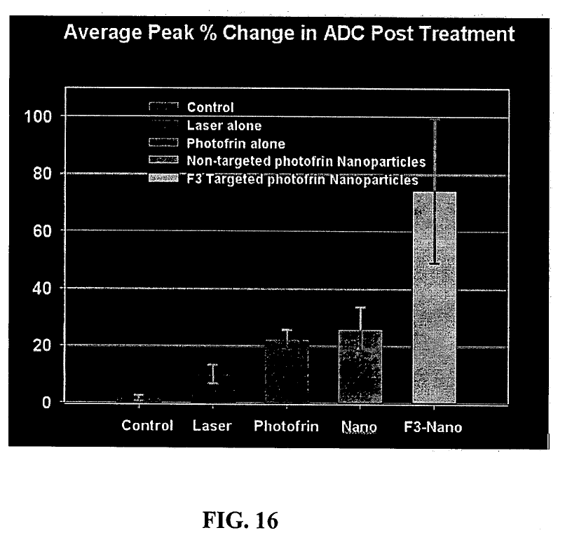

All treatment groups were quantitated and their diffusion value changes are displayed in FIG. 16. Laser-alone exposure had a very small increase in mean tumor diffusion values over control animals. Photofrin and Photofrin-containing nanoparticle treated tumors had a 21 to 24% increase in tumor ADC values. It is very interesting to note that the change in ADC values of these two groups of treated tumors were similar in magnitude indicating that the two treatments had similar cell killing capacity. However, Photofrin/F3-Nanoparticles were found to produce the most significant increase in tumor ADC values which should in principle translate into the most improved animal survival. FIG. 16 shows a bar graph of the average % peak increase in tumor ADC values following therapeutic intervention for control, laser only, Photofrin, Photofrin-containing nanoparticles and Photofrin/F3-nanoparticles. Note that the most significant increase in peak ADC percentage was for the F3-targeted nanoparticles.

Kaplan-Meier survival statistics were obtained on these 5 groups of animals in order to assess therapeutic efficacy. As shown in FIG. 17, control and laser treated animals were found to have no significant differences in survival times. This was also found to be true for Photofrin administered via tail vein injection and with nanoparticle-encapsulated Photofrin administered via tail vein. This result reveals that the nanoparticle delivery vehicle could be engineered to produce the same therapeutic benefit as the “free” drug but without the potential systemic side effects such as photosensitization of the skin and eyes. Results obtained from treatment with Photofrin-encapsulated F3-targeted nanoparticles indicated even greater survival times. Treatment of rats with the F3-targeted nanoparticles resulted in 3 out of 5 tumor cures. These results reveal the viability of this approach for cancer treatment. FIG. 17 shows Kaplan-Meier survival statistics for control, laser only, photofrin, Photofrin-containing nanoparticles and Photofrin/F3-nanoparticles. Note that the most significant increase in animal survival was for the Photofrin/F3-nanoparticle treated group of animals wherein 3 out of 5 animals treated were cured of tumor burden.

The above results demonstrate the feasibility of targeted delivery of a therapeutic agent in a tumor-specific manner. In summary, these in vivo experiments have demonstrated the ability to synthesize nanoparticles that have the ability to target, in a tumor-specific manner, and thus deliver a therapeutic agent to the tumor. When the therapeutic agent is Photofrin, the majority of tumors treated using this nanoparticle approach were cured. Furthermore, extension of the approach into other therapeutic agents such as radiation sensitizers has also shown significant efficacy.

### Example 49

Radio Sensitization

Because Photofrin, the therapeutic agent used in PDT, requires an external source of light to activate its therapeutic potential, it was desirable to investigate if a therapeutic agent such as Gemcitabine could be better suited since the synergistic enhancement of therapeutic efficacy can be accomplished using ionizing radiation as the activator. Unlike PDT, ionizing radiation can activate the therapeutic agent without invasive, surgical procedures. To evaluate this, preliminary experiments were accomplished using radiosensitizer-containing or bound nanoparticles.

In Vivo Studies

Preliminary experiments were accomplished using F3-targeted Gemcitabine-containing nanoparticles. Animals were treated with either ionizing radiation (I.R.) alone, Gemcitabine (i.v.) or F3-targeted Gemcitabine-containing nanoparticles (i.v.). Diffusion MRI as a surrogate marker of cell kill was accomplished over time as shown in FIG. 18.

It can be noted that the F3-targeted Gemcitabine I.R.-treated tumors had the largest increase in tumor ADC values. This reveals that targeted nanoparticle delivery of the radiosensitizer agent (Gemcitabine) to the tumor site produced the greatest sensitization of the tumor mass to ionizing radiation (I.R.). FIG. 18 shows ADC overlay maps over time (days) for ionizing radiation (5 Gy/day×5 days), for ionizing radiation (5 Gy/day×5 days) plus Gemcitabine (i.v.), and I.R.+Gemcitabine-containing F3-targeted nanoparticles (bottom two animals). Note: Compare results for day 10.

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

1. A polymeric nanoparticle comprising:
(a) a backbone polymer selected from the group consisting of poly(acrylamide), poly(2-hydroxyethyl methacrylate), poly(glycerol monomethacrylate), poly(acrylic acid), poly (aminoalkyl)methacrylamide), poly(sodium acrylate), poly(vinyl pyrrolidone) and mixtures thereof; and
(b) a polymeric cross-linker selected from the group consisting of glycerol(bis)acrylate, 3-(acryloyloxy)-2-hydroxypropyl methacrylate, ethylene glycol diacrylate, glycerol dimethacrylate, divinyl citrate, and mixtures thereof,
wherein said polymeric cross-linker links two or more of said backbone polymers.

2. The polymeric nanoparticle of claim 1, wherein the nanoparticle is biodegradable.

3. The polymeric nanoparticle of claim 1, wherein said backbone polymer is poly(acrylamide).

4. The polymeric nanoparticle of claim 1, wherein said backbone polymer is poly(3-(aminopropyl)methacrylamide).

5. The polymeric nanoparticle of claim 1, wherein said backbone polymer is poly(acrylamide) and said polymeric cross-linker is glycerol(bis) acrylate.

6. The polymeric nanoparticle of claim 1, wherein said backbone polymer is poly(vinyl pyrrolidone) and said polymeric cross-linker is divinyl citrate.

7. The polymeric nanoparticle of claim 1, further comprising a functionalized surface group.

8. The polymeric nanoparticle of claim 1 or claim 7, wherein the nanoparticle encapsulates one or more water-soluble agents.

9. The polymeric nanoparticle of claim 8, wherein said water-soluble agent is selected from the group consisting of a small organic molecule drug, a DNA molecule, an RNA molecule, a protein, a fluorescent dye, a radioisotope, a contrast agent, a degradable polymer and an imaging agent.

10. The polymeric nanoparticle of claim 8, wherein the nanoparticle encapsulates two or more water-soluble agents.

11. The polymeric nanoparticle of claim 1 or claim 7, wherein the nanoparticle encapsulates one or more water-insoluble agents.

12. The polymeric nanoparticle of claim 8, wherein the water-soluble agent is photofrin.

13. The polymeric nanoparticle of claim 8, wherein the water-soluble agent is iron oxide.

14. The polymeric nanoparticle of claim 8, wherein the water-soluble agent is gemcitabine.

15. The polymeric nanoparticle of claim 7, wherein said functionalized surface group is an amine group.

16. The polymeric nanoparticle of claim 7, wherein said functionalized surface group is bound to an antibody molecule.

17. The polymeric nanoparticle of claim 7, wherein said functionalized surface group is conjugated to a peptide.

18. The polymeric nanoparticle of claim 7, wherein said functionalized surface group is conjugated to Herceptin.

19. The polymeric nanoparticle of claim 7, wherein said functionalized surface group is conjugated to a peptide selected from the group consisting of SEQ ID NO. 1, SEQ ID NO: 2 and SEQ ID NO: 3.

20. The polymeric nanoparticle of claim 7, wherein said functionalized surface group is conjugated to the peptide represented in SEQ ID NO. 3.

21. The polymeric nanoparticle of claim 1, wherein the nanoparticle is less than 200 nm in diameter.

22. The polymeric nanoparticle of claim 1, wherein the nanoparticle is less than 100 nm in diameter.

23. The polymeric nanoparticle of claim 1, wherein said backbone polymer is poly(acrylamide) and said polymeric cross-linker is 3-(acryloyloxy)-2-hydroxypropyl methacrylate.

24. The polymeric nanoparticle of claim 1, wherein said backbone polymer is a mixture of poly(acrylamide) and poly(3-(aminopropyl)methacrylamide) and said polymeric cross-linker is 3-(acryloyloxy)-2-hydroxypropyl methacrylate.

25. The polymeric nanoparticle of claim 1, wherein said backbone polymer is a mixture of poly(acrylamide) and poly(acrylic acid) and said polymeric cross-linker is 3-(acryloyloxy)-2-hydroxypropyl methacrylate.

26. The polymeric nanoparticle of claim 1, wherein said backbone polymer is a mixture of poly(acrylamide) and poly(3-(aminopropyl)methacrylamide) and said polymeric cross-linker is glycerol(bis)acrylate.

27. The polymeric nanoparticle of claim 1, wherein said backbone polymer is a mixture of poly(acrylamide) and poly(acrylic acid) and said polymeric cross-linker is glycerol(bis)acrylate.

28. The polymeric nanoparticle of claim 24, wherein said nanoparticle is surface functionalized and conjugated to the peptide represented in SEQ ID NO:3, and wherein said nanoparticle encapsulates gemcitabine.

29. The polymeric nanoparticle of claim 24, wherein said nanoparticle is surface functionalized and conjugated to the peptide represented in SEQ ID NO:3, and wherein said nanoparticle encapsulates gemcitabine and iron oxide.

30. The polymeric nanoparticle of claim 24, wherein said nanoparticle is surface functionalized and conjugated to the peptide represented in SEQ ID NO:3, and wherein said nanoparticle encapsulates gemcitabine and PLGA.

31. The polymeric nanoparticle of claim 10, wherein a first water-soluble agent is a degradable polymer, selected from the group consisting of PLGA, polysorbitol, polysorbitol-adipate, polymannitol, polyaspartic acid, polylysine, polyglutamic acid; and wherein a second agent is selected from the group consisting of a small organic molecule drug, a DNA molecule, an RNA molecule, a protein, a fluorescent dye, a radioisotope, a contrast agent, and an imaging agent.

32. A method of producing a polymeric nanoparticle comprising:
(a) forming a solution of polymeric monomers and cross-linkers, the monomers selected from the group consisting of acrylamide, (aminoalkyl)methacrylamide, 2-hydroxyethyl methacrylate, glycerol monomethacrylate, acrylic acid, sodium acrylate, vinyl pyrrolidone, and mixtures thereof; the cross-linkers selected from the group consisting of glycerol(bis) acrylate, 3-(acryloyloxy)-2-hydroxypropyl methacrylate, ethylene glycol diacrylate, glycerol dimethacrylate, divinyl citrate and mixtures thereof;
(b) initiating polymerization to generate a solid particle, the particle comprising a polymeric backbone of the polymeric monomers cross-linked with the polymeric cross-linker; and
(c) removing the solid particle from solution.

33. The method of claim 32, further comprising passing the solid particle through one or more porous filters to generate a nanoparticle that is less than 200 nm in diameter.

34. The method of claim 32, further comprising adding an agent to be encapsulated to the solution prior to said initiation (b).

35. The method of claim 34, wherein said agent is a water-soluble agent.

36. The method of claim 32, further comprising adding in (a) a functionalized monomer, thereby generating a functionalized group on the surface of the nanoparticle.

37. The method of claim 32, further comprising adding a surfactant to the solution prior to said initiation (b).

38. The method of claim 32, wherein the polymeric monomer is 3-(aminopropyl)methacrylamide.

39. A method of treating a tumor in a mammalian patient comprising:
(a) administering to the patient a polymeric nanoparticle according to claim 1,
wherein the polymeric nanoparticle encapsulates one or more cancer chemotherapeutic agents.

40. The method of claim 39, wherein the cancer chemotherapeutic agent is selected from the group consisting of gemcitabine and photofrin.

41. The method of claim 39, wherein the nanoparticle further encapsulates an imaging agent.

42. The method of claim 41, wherein the imaging agent is iron oxide.

43. The method of claim 41, further comprising imaging the polymeric nanoparticle in the patient.

44. A method of treating a tumor in a mammalian patient comprising:
(a) administering to the patient a polymeric nanoparticle according to claim 1; and
(b) administering ionizing radiation to the patient,
wherein the polymeric nanoparticle encapsulates one or more radiation-sensitizing agents.

45. The method of claim 44, wherein the radiation-sensitizing agent is selected from the group consisting of gemcitabine, paclitaxel and carboplatin.

46. The method of claim 44, wherein the nanoparticle further encapsulates an imaging agent.

47. The method of claim 46, wherein the imaging agent is iron oxide.

48. The method of claim 46, further comprising imaging the polymeric nanoparticle in the patient.

49. A method of imaging a polymeric nanoparticle in a mammalian patient comprising:
(a) administering to the patient a polymeric nanoparticle according to claim 1; and
(b) imaging the nanoparticle,
wherein the polymeric nanoparticle encapsulates one or more imaging agents.

50. The method of claim 49, wherein the imaging agent is iron oxide.

51. A polymeric nanoparticle comprising:
(a) a backbone polymer selected from the group consisting of poly(acrylamide), poly(acrylic acid), poly(3-(aminopropyl)methacrylamide) and mixtures thereof, cross-linked with;
(b) about 10% glycerol(bis)acrylate cross-linker; and
(c) a functionalized surface group conjugated to a peptide selected from the group consisting of SEQ ID NO: 1, SEQ ID NO: 2 and SEQ ID NO:3,
wherein said polymeric nanoparticle encapsulates iron oxide.

52. The polymeric nanoparticle of claim 51, further encapsulating gemcitabine.

53. A polymeric nanoparticle comprising:
(a) a backbone polymer selected from the group consisting of poly(acrylamide), poly(acrylic acid), poly(3-(aminopropyl)methacrylamide) and mixtures thereof, cross-linked with;
(b) about 20% glycerol(bis)acrylate cross-linker; and
(c) a functionalized surface group conjugated to a peptide selected from the group consisting of SEQ ID NO: 1, SEQ ID NO: 2 and SEQ ID NO:3,
wherein said polymeric nanoparticle encapsulates iron oxide.

54. The polymeric nanoparticle of claim 53, further encapsulating gemcitabine.

55. A polymeric nanoparticle comprising:
(a) a backbone polymer selected from the group consisting of poly(acrylamide), poly(acrylic acid), poly(3-(aminopropyl)methacrylamide) and mixtures thereof, cross-linked with;
(b) about 10% 3-(acryloyloxy)-2-hydroxypropyl methacrylate cross-linker; and
(c) a functionalized surface group conjugated to a peptide selected from the group consisting of SEQ ID NO: 1, SEQ ID NO: 2 and SEQ ID NO:3,
wherein said polymeric nanoparticle encapsulates iron oxide.

56. The polymeric nanoparticle of claim 55, further encapsulating gemcitabine.

57. A polymeric nanoparticle comprising:
(a) a backbone polymer selected from the group consisting of poly(acrylamide), poly(acrylic acid), poly(3-(aminopropyl)methacrylamide) and mixtures thereof, cross-linked with;
(b) about 20% 3-(acryloyloxy)-2-hydroxypropyl methacrylate cross-linker; and
(c) a functionalized surface group conjugated to a peptide selected from the group consisting of SEQ ID NO: 1, SEQ ID NO: 2 and SEQ ID NO:3,
wherein said polymeric nanoparticle encapsulates iron oxide.

58. The polymeric nanoparticle of claim 57, further encapsulating gemcitabine.

59. A polymeric nanoparticle produced by the process comprising:
(a) forming a solution of polymeric monomers and cross-linkers, the monomers selected from the group consisting of acrylamide, 3-(aminopropyl)methacrylamide, acrylic acid, and mixtures thereof; the cross-linkers selected from the group consisting of glycerol(bis)acrylate, 3-(acryloyloxy)-2-hydroxypropyl methacrylate and mixtures thereof;
(b) optionally adding a functionalized monomer;
(c) adding iron oxide;
(d) initiating polymerization to generate a solid particle, the particle comprising a polymeric backbone of the polymeric monomers cross-linked with the polymeric cross-linker;
(e) conjugating a peptide selected from the group consisting of SEQ ID NO: 1, SEQ ID NO: 2 and SEQ ID NO:3 to a functionalized surface group on the nanoparticle; and
(f) removing the nanoparticle from solution,
wherein the cross-linker density is about 10% relative to the polymeric backbone.

60. The polymeric nanoparticle produced by the process of claim 59, further comprising adding gemcitabine prior to (d).

61. A polymeric nanoparticle produced by the process comprising:
(a) forming a solution of polymeric monomers and cross-linkers, the monomers selected from the group consisting of acrylamide, 3-(aminopropyl)methacrylamide, acrylic acid, and mixtures thereof; the cross-linkers selected from the group consisting of glycerol(bis)acrylate, 3-(acryloyloxy)-2-hydroxypropyl methacrylate and mixtures thereof;
(b) optionally adding a functionalized monomer;
(c) adding iron oxide;
(d) initiating polymerization to generate a solid particle, the particle comprising a polymeric backbone of the polymeric monomers cross-linked with the polymeric cross-linker;
(e) conjugating a peptide selected from the group consisting of SEQ ID NO: 1, SEQ ID NO: 2 and SEQ ID NO:3 to a functionalized group on the nanoparticle; and
(f) removing the nanoparticle from solution,
wherein the cross-linker density is about 20% relative to the polymeric backbone.

62. The polymeric nanoparticle produced by the process of claim 61, further comprising adding gemcitabine prior to (d).
